(19) United States
(12) Patent Application Publication
(10) Pub. No.: US 2026/0175020 A1
(43) Pub. Date: Jun. 25, 2026

(54) CONFIGURATIONS OF ELONGATED BODY FOR ENDOVASCULAR THERAPY SYSTEM

(71) Applicant: Covidien LP, Mansfield, MA (US)

(72) Inventors: Dale F. Seeley, Spring Park, MN (US); Robert L. Olson, White Bear Lake, MN (US)

(21) Appl. No.: 19/426,817

(22) Filed: Dec. 19, 2025

Related U.S. Application Data
(60) Provisional application No. 63/736,384, filed on Dec. 19, 2024.

(51) Int. Cl. A61N 1/05; A61N 1/36; A61N 1/375

(52) U.S. Cl. CPC A61N 1/0551; A61N 1/36053; A61N 1/37516; A61N 1/37518

(57) ABSTRACT

An endovascular therapy system includes an endovascular device. The endovascular device includes an elongated body configured to be introduced into vasculature of a patient. The endovascular device includes an expandable structure at a distal portion of the elongated body. The therapy system (e.g., the endovascular device) can include a plurality of electrodes carried by the expandable structure. The elongated body can be configured to transform between a body delivery configuration and a body deployed configuration. In the body deployed configuration, the distal portion of the elongated body defines a coiled portion. The coiled portion can be located proximal of the expandable structure. The coiled portion is configured to limit or prevent an axial force applied to the elongated body from being transferred to the expandable structure or the plurality of electrodes when the expandable structure and the elongated body are positioned within the vasculature of the patient.

### CROSS-REFERENCE TO RELATED APPLICATIONS

This application claims the benefit of and priority to U.S. Provisional Patent Application Serial No. 63/736,384 filed December 19, 2024, the entire disclosure of which is incorporated by reference herein.

### TECHNICAL FIELD

This disclosure relates to electrical stimulation therapy.

### BACKGROUND

Medical devices, such as electrical stimulation devices, may be used in different therapeutic applications, such as vagus nerve stimulation (VNS) and/or deep brain stimulation (DBS). A medical device may be used to deliver therapy to a patient to treat a variety of symptoms or patient conditions. In some therapy systems, an external or an implantable electrical stimulator delivers electrical stimulation therapy to a target tissue site within a patient with the aid of one or more electrodes. Additionally or alternatively, a medical device senses one or more patient parameters with the aid of the one or more electrodes.

### SUMMARY

This disclosure describes medical devices systems (e.g., endovascular therapy systems) including endovascular devices configured for endovascular delivery of electrical stimulation therapy to a patient (e.g., to one or more nerves or brain targets) and/or sensing of one or more patient parameters (e.g., nerve signals, brain signals, and/or other physiological parameters), and related methods. In particular, this disclosure describes configurations for structures of endovascular therapy systems that facilitate delivery of electrical stimulation therapy and/or sensing of patient parameters from an endovascular location.

An endovascular device can include an elongated body (e.g., a medical lead) and an expandable structure carrying one or more electrodes. In some cases, the elongated body (e.g., medical lead) and/or the expandable structure can become endothelialized (e.g., become integrated with and/or into the endothelium of the blood vessel). In some cases, endothelialization can fix the position of the expandable structure and/or the electrodes relative to the blood vessel and/or target tissue surrounding the blood vessel. Thus, movement of electrodes relative to target tissue occurring over relatively longer periods of time can be reduced, mitigated, or even prevented when adequate endothelialization occurs. However, prior to, and/or in the absence of, adequate endothelization in which the expandable structure and/or the electrodes become incorporated into the vessel wall of the blood vessel, movement of the elongated body (e.g., the medical lead) can in some cases transfer forces to the expandable structure, which can cause the expandable structure and/or the electrodes to move (e.g., move proximally, distally, and/or rotationally) relative to the target tissue outside of the blood vessel.

In one or more examples, the elongated body (e.g., which may be, include, and/or be a part of a medical lead) is configured to accommodate axial forces (e.g., forces that tend to push or pull on the elongated body), such that the transfer of such forces to the expandable structure, and thus also the electrodes carried by the expandable structure, is reduced or even eliminated. For example, the elongated body may include one or more coiled portions positioned proximal to the expandable structure and/or the electrodes carried by the expandable structure. The coiled portions can be configured to anchor the elongated body in vasculature of the patient. In some examples, the one or more coiled portions of the elongated body are configured to absorb forces applied to the elongated body, e.g., axial forces, rotational force, and/or twisting forces, while minimizing or even eliminating such forces from being transferred to the expandable structure and/or the electrodes.

In some examples, an endovascular device includes an elongated body configured to be introduced into vasculature of a patient; an expandable structure at a distal portion of the elongated body; and a plurality of electrodes carried by the expandable structure, wherein the elongated body is configured to transform between a body delivery configuration and a body deployed configuration, and wherein in the body deployed configuration, the distal portion of the elongated body defines a coiled portion, the coiled portion located proximal of the expandable structure and configured to limit or prevent an axial force applied to the elongated body from being transferred to the expandable structure or the plurality of electrodes.

In some examples, a method includes introducing an endovascular device into vasculature of a patient, the endovascular device includes an elongated body configured to be introduced into the vasculature of the patient; an expandable structure at a distal portion of the elongated body; and a plurality of electrodes carried by the expandable structure, wherein the elongated body is configured to transform between a body delivery configuration and a body deployed configuration, and wherein in the body deployed configuration, the distal portion of the elongated body defines a coiled portion, the coiled portion located proximal of the expandable structure and configured to limit or prevent an axial force applied to the elongated body from being transferred to the expandable structure or the plurality of electrodes; and advancing the endovascular device until the plurality of electrodes are at or near a target location in the vasculature of the patient.

In some examples, an endovascular device includes an elongated body configured to be introduced into vasculature of a patient; an expandable structure at a distal portion of the elongated body; a plurality of electrodes carried by the expandable structure; and two or more anchors positioned on a coiled portion of the elongated body, each anchor of the two or more anchors configured to engage a vessel wall of the vasculature when the elongated body is positioned within the vasculature of the patient, wherein the elongated body is configured to transform between a body delivery configuration and a body deployed configuration, and wherein in the body deployed configuration, the distal portion of the elongated body defines the coiled portion, the coiled portion located proximal of the expandable structure and configured to limit or prevent an axial force applied to the elongated body from being transferred to the expandable structure or the plurality of electrodes.

The examples described herein may be combined in any permutation or combination.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a conceptual diagram illustrating an example endovascular therapy system including an endovascular device configured for delivering electrical stimulation therapy to a target tissue site of a patient and/or for sensing a patient parameter from an endovascular location.

FIG. 2 is a functional block diagram illustrating components of an example medical device of the therapy system of FIG. 1.

FIG. 3A illustrates a distal portion of an example endovascular therapy system including an elongated body, an expandable structure at a distal portion of the elongated body, and electrodes carried by the expandable structure.

FIG. 3B illustrates a distal portion of an example endovascular therapy system including an elongated body and without an expandable structure or electrodes.

FIG. 3C illustrates a cross-sectional view of a portion of the elongated body of FIG. 3B.

FIG. 3D illustrates of an example endovascular device positioned within vasculature of a patient.

FIG. 3E illustrates a cross-sectional view of a portion of the endovascular device positioned within vasculature of FIG. 3D.

FIG. 4 illustrates an elongated body including a first coiled portion and a second coiled portion.

FIG. 5 is a flow diagram illustrating an example technique for introducing and advancing an endovascular device according to this disclosure.

Like reference characters denote like elements throughout the description and figures.

### DETAILED DESCRIPTION

This disclosure describes devices, systems, and methods relating to delivery of electrical stimulation therapy, such as vagus nerve stimulation (VNS), deep brain stimulation (DBS), and/or sensing one or more patient parameters (e.g., nerve activity from one more nerves, cardiac signals, muscle activation signals, brain signals and/or other physiological parameters, such as impedance, electroencephalogram (EEG), evoked potentials, local field potentials, etc.) from an endovascular location. Example endovascular locations that can be used for electrical stimulation therapy (e.g., VNS therapy) and/or sensing using the devices described herein include an internal jugular vein (IJV). Example endovascular locations that can be used to access the brain sites for electrical stimulation therapy (e.g., DBS) and/or sensing using the devices described herein include any suitable cranial blood vessel (also referred to herein as a cerebral blood vessel or neurovasculature, which can include a vein or an cranial artery), such as, but not limited to, the thalamostriate vein, the internal cerebral vein, the basal vein of Rosenthal, the inferior sagittal sinus, the superior sagittal sinus, or the anterior choroidal artery.

VNS has been proposed for use to manage one or more patient conditions, such as to control an inflammatory response in patients. Stimulating the vagus nerve may dampen the inflammatory response and associated cytokine response. In some examples, inflammatory cytokines are modulated up or down via stimulation. In addition, VNS may assist in stroke rehabilitation and limit ischemia reperfusion injury. After a myocardial infarct or stroke, reperfusion therapies (surgery or drugs) are given to restore blood flow. However, due to the restoration of blood, flow induced local damage occurs, including ischemia reperfusion injury. This injury may induce local accumulations of chemical mediators such as reactive oxygen species (ROS) production, inflammatory cytokines, bradykinin, etc., which can further affect inflammation. Such inflammatory compounds may trigger sensory signaling, which can lead to a reduced organ vagus activity and sympathetic overdrive. Vagus nerve stimulation may treat reperfusion damage as the inflammatory state may be lowered by increasing parasympathetic drive.

DBS has been proposed for use to manage one or more patient conditions. For example, DBS can be used to alleviate, and in some cases, eliminate symptoms associated with movement disorders, other neurodegenerative impairment, seizure disorders, psychiatric disorders (e.g., mood disorders), or the like. Movement disorders may be found in patients with Parkinson’s disease, multiple sclerosis, and cerebral palsy, among other conditions, and can be associated with disease or trauma. DBS can be delivered to one or more target sites in a brain of a patient to help a patient with muscle control and minimize movement problems, such as rigidity, bradykinesia (i.e., slow physical movement), rhythmic hyperkinesia (e.g., tremor), nonrhythmic hyperkinesia (e.g., tics) or akinesia (i.e., a loss of physical movement).

In the case of seizure disorders, DBS can be delivered to one or more target sites in a brain of a patient to reduce the frequency or severity of seizures, or even help prevent the occurrence of seizures. In the case of psychiatric disorders, DBS can be delivered to help minimize or even eliminate symptoms associated with major depressive disorder (MDD), bipolar disorder, anxiety disorders, post-traumatic stress disorder, dysthymic disorder, or obsessive-compulsive disorder (OCD). DBS can also reduce the symptoms of Parkinson’s disease, dystonia, or cerebellar outflow tremor.

While this disclosure describes examples of VNS and/or sensing via applicable endovascular locations (e.g., the internal jugular vein), it should be understood that the devices, systems, and techniques may be adapted for DBS, other kinds of brain stimulation, peripheral nerve stimulation, or electrical stimulation and/or sensing of any nerve tissue that can be done via an endovascular location.

In one or more examples, an endovascular therapy system includes an endovascular device. The endovascular device can include one or more electrodes and/or other sensing elements that are carried by an expandable structure at a distal portion of an elongated body of the endovascular device (e.g., an elongated body of a medical lead). The expandable structure (e.g., a stent, or stent-like structure) is configured to transform between a delivery (e.g., compressed or relatively low-profile) configuration and a deployed (e.g., expanded) configuration. One or more electrodes and/or sensing elements are mechanically coupled to, disposed on, or otherwise carried by the expandable structure. Each electrode and/or sensing element is electrically connected to a medical device via one or more conductor wires. In some examples, the medical device is configured to deliver therapy (e.g., electrical stimulation therapy) and/or received sensed signals via the electrodes by way of the conductor wires. The conductor wires can extend from the medical device, along the elongated body, and to each electrode to electrically connect to each electrode or sensing element.

In some cases, the endovascular therapy systems herein are configured to deliver electrical stimulation therapy to target tissue (e.g., one or more nerves) located outside of (e.g., radially outside of) a blood vessel in which the electrodes are positioned. Additionally or alternatively, the endovascular therapy systems herein are configured to receive signals (e.g., bioelectric signals) from the one or more nerves located outside of the blood vessel in which the electrodes and/or sensing elements are positioned.

In some cases, the positioning of the electrodes relative to the target location, such as relative to one or more nerves located radially outside of the blood vessel in which the electrodes are positioned, can affect the efficacy of therapy, e.g., electrical stimulation therapy, delivered to the one or more nerves. For example, electrodes can be placed at an axial location within a blood vessel where the target tissue (e.g., a target nerve) is relatively close to the blood vessel as compared to other axial locations of the blood vessel. In some cases, the closeness (e.g., physical proximity) of a nerve or other target tissue to a blood vessel (e.g., as measured in a radial direction outward from the blood vessel) changes along an axial direction of the blood vessel. In some cases, efficacy of therapy (e.g., the effect of electrical stimulation therapy) and/or the ability to sense (e.g., ability to detect electrical signals) changes with the closeness (e.g., physical proximity) of the nerve or other target tissue to the blood vessel. In some cases, stimulation therapy is relatively more effective and/or relatively easier (e.g., due to less required electrical energy) in instances where target tissue is relatively close to the blood vessel (e.g., as measured in the radial direction outward of the blood vessel).

In some cases, the elongated body (e.g., medical lead) and/or the expandable structure can become endothelialized (e.g., become integrated with and/or into the endothelium of the blood vessel). In some cases, endothelialization can fix the position of the expandable structure and/or the electrodes relative to the blood vessel and/or target tissue surrounding the blood vessel. Thus, movement of electrodes relative to target tissue occurring over relatively longer periods of time can be reduced, mitigated, or even prevented when adequate endothelialization occurs. However, prior to, and/or in the absence of, adequate endothelization in which the expandable structure and/or the electrodes become incorporated into the vessel wall of the blood vessel, movement of the elongated body (e.g., the medical lead) can in some cases transfer forces to the expandable structure, which can cause the expandable structure and/or the electrodes to move (e.g., move proximally, distally, and/or rotationally) relative to the target tissue outside of the blood vessel.

In one or more examples, the elongated body (e.g., which may be, include, and/or be a part of a medical lead) is configured to accommodate axial forces (e.g., forces that tend to push or pull on the elongated body), such that the transfer of such forces to the expandable structure, and thus also the electrodes carried by the expandable structure, is reduced or even eliminated. Additionally or alternatively, in one or more examples, the elongated body is configured to accommodate rotational forces and/or twisting forces (e.g., forces that tend cause the elongated body to rotate or twist, such as relative to the blood vessel), such that the transfer of such forces to the expandable structure, and thus also the electrodes carried by the expandable structure, is reduced or even eliminated. For example, the elongated body can include and/or define one or more structural features that enable the elongated body to absorb forces while minimizing or even eliminating the transfer of such forces to the expandable structure. Such minimization and/or elimination of forces to the expandable structure can, in turn, limit undesirable movement of the expandable structure and/or the electrodes relative to the blood vessel.

The elongated body may include one or more coiled portions. The one or more coiled portions of the elongated body may be positioned proximal to the expandable structure and/or the electrodes carried by the expandable structure. The coiled portions can be configured to anchor the elongated body in vasculature of the patient. In some examples, the one or more coiled portions of the elongated body are configured to absorb forces applied to the elongated body, e.g., axial forces, rotational force, and/or twisting forces, while minimizing or even eliminating such forces from being transferred to the expandable structure and/or the electrodes. This minimization and/or elimination of forces to the expandable structure can limit and/or prevent movement of the electrodes relative to target tissue (e.g., target tissue radially outside of the blood vessel). Such minimization and/or elimination of forces prior to adequate endothelialization and/or in the absence of adequate endothelialization can ensure that the expandable structure and/or the electrodes remain in a relatively stable location within the vasculature of a patient.

When the elongated body includes multiple coiled portions, each coiled portion of the multiple coiled portions may be configured to anchor the elongated body within the vasculature. In some examples, each coiled portion of the multiple coiled portions is configured to be positioned in a different portion of the vasculature of the patient. For example, a first coiled portion can be configured to be positioned in a relatively distal portion of the vasculature (e.g., in a first blood vessel, such as a jugular vein or a carotid artery). A second coiled portion (e.g., different from the first coiled portion) can be configured to be positioned in a more proximal portion of the vasculature (e.g., in a second blood vessel, such as a subclavian vein or a subclavian artery). In some cases, having multiple, spaced apart coiled portions of the elongated body can enable relatively better anchoring in the vasculature, e.g., as compared to examples in which the elongated body only includes one or no coiled portions. For example, each respective coiled portion of the multiple coiled portions can be sized, shaped, and/or otherwise configured according to the respective blood vessel in which each respective coiled portion is intended to reside, which can help facilitate relatively better anchoring of the elongated body as a whole within the vasculature.

The elongated body may include one or more additional structures that serve as additional and/or complimentary features to the coiled portions. For example, the elongated body includes one or more anchors. In some examples, the anchors are configured to help anchor the elongated body within the vasculature of the patient. The anchors may be mechanically coupled to and/or integrally formed with a portion of the elongated body. In some examples, the one or more anchors are positioned on one or more of the coiled portions of the elongated body. The anchors may be configured to contact, be in close proximity to, and/or otherwise engage a blood vessel wall. The anchors can serve as additional and/or complementary structural features to the one or more coiled portions of the elongated body. In some examples, the anchors are configured to minimize or even prevent forces applied to the elongated body, e.g., axial forces, rotational forces, and/or twisting forces, from being transferred to the expandable structure and/or to the electrodes.

In some examples, a medical device is configured to generate electrical stimulation and/or sense a patient parameter via the electrodes of the endovascular device. The electrodes may be carried by or otherwise disposed on an expandable structure, which may be configured to orient the electrodes and/or anchor the electrodes at a particular location in the vasculature of the patient.

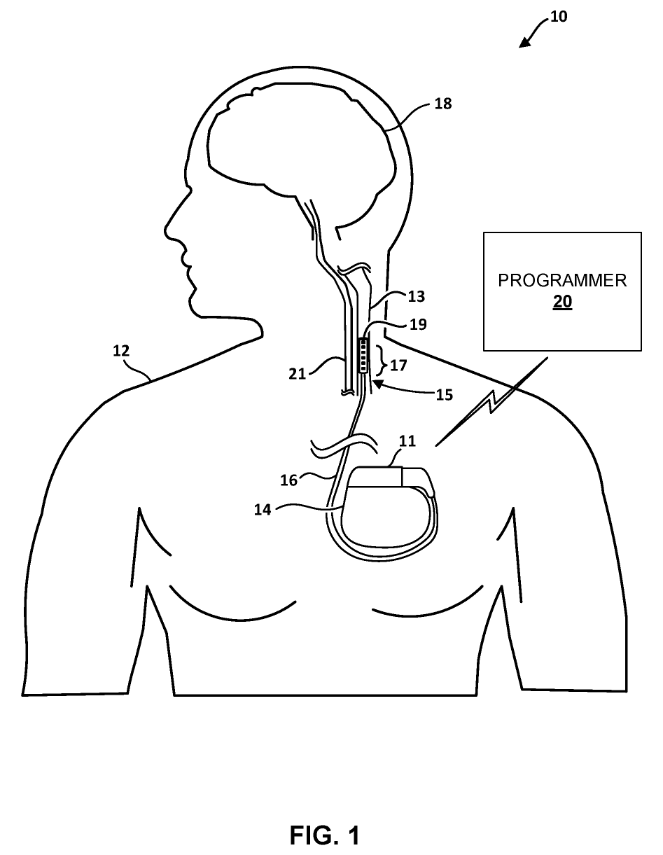

FIG. 1 is a conceptual diagram illustrating an example therapy system 10 configured to deliver electrical stimulation therapy to a target tissue site of a patient 12 or sense a patient parameter from an endovascular location. Patient 12 ordinarily will be a human patient. In some cases, however, therapy system 10 is applied to other mammalian or non-mammalian non-human patients. Therapy system 10 includes a medical device 14 and an endovascular device 16. In the example of FIG. 1, medical device 14 is configured to deliver electrical stimulation therapy (e.g., VNS) to a vagus nerve 21 of patient 12 and/or sense bioelectric signals via electrodes 17. However, in other examples, therapy system 10 and/or medical device 14 is configured to deliver electrical stimulation therapy (e.g., DBS) to brain 18 of patient 12 and/or sense bioelectrical brain signals in brain 18 via electrodes 17.

In the example of FIG. 1, endovascular device 16 is positioned in a jugular vein 13 of patient 12 such that one or more electrodes 17 are located proximate to a target tissue site. In particular, electrodes 17 are positioned to deliver electrical stimulation therapy to and/or sense signals from nerves surrounding jugular vein 13, including (but not limited to) vagus nerve 21. Jugular vein 13 can include one or more of the external jugular vein, the internal jugular vein, or the anterior jugular vein. In some examples, endovascular device 16 can additionally or alternatively be positioned in a carotid artery. Endovascular device 16 includes an expandable structure 19 at a distal portion 15 of endovascular device 16 which may help hold electrodes 17 in apposition with a vessel wall (e.g., of jugular vein 13). In some examples, expandable structure 19 is mechanically coupled (e.g., directly mechanically coupled) to a portion of endovascular device 16 via a suitable mechanical connection (e.g., welding, crimped connection, or the like). Medical device 14 can provide electrical stimulation to one or more regions surrounding jugular vein 13 in order to manage a condition of patient 12, such as to mitigate the severity or duration of the patient condition.

In some examples, a portion of endovascular device 16 (e.g., a proximal portion of endovascular device 16) can be positioned in a subclavian vein and/or a subclavian artery. For example, endovascular device 16 enters the vasculature through a subclavian vein and/or a subclavian artery and navigated to a more distal portion of the vasculature (e.g., jugular vein 13). In such an example, distal portion 15 of endovascular device 16 is positioned in jugular vein 13 and another portion of endovascular device 16 (e.g., a portion of endovascular device 16 proximal to distal portion 15) can remain positioned in the subclavian vein.

As discussed further in relation to examples herein, endovascular device 16 includes and/or one or more structural features configured to limit or prevent a force (e.g., an axial force, rotational force, twisting force, and/or the like) applied to endovascular device 16 from being transferred to expandable structure 19 and/or the electrodes 17. Such forces can be the result of various physiological functions of patient 12, such as from movement of patient 12 (e.g., normal movement, breathing, neck turning, standing up, sitting down, and/or the like). Additionally or alternatively, such forces applied to endovascular device 16 that would otherwise cause movement of expandable structure 19 can result from forces placed on endovascular device 16 during a surgical procedure in which endovascular device 16 is placed within patient 12 (e.g., due to retraction of a delivery catheter relative to endovascular device 16, due to connecting a proximal end of endovascular device 16 to medical device 14, and/or the like).

In some examples, endovascular device 16 includes and/or defines one or more coiled portions. For example, the body of endovascular device 16 can define one or more loops that form a coil shape. In some examples, distal portion 15 of endovascular device 16 defines the one or more coiled portions. The one or more coiled portions can help anchor endovascular device 16 in the vasculature of patient 12 (e.g., within one or more vessels such as jugular vein 13, a subclavian vein, and/or another vein or artery). The coiled portions can limit or prevent one or more loads and/or forces from being transmitted to expandable structure 19 and/or electrodes 17. Limiting and/or preventing one or more loads and/or forces from being transmitted to expandable structure 19 and/or electrodes 17 can limit and/or prevent expandable structure 19 and/or electrodes 17 from moving relative to a target (e.g., desired) location. By keeping expandable structure 19 and/or electrodes 17 in a relatively stable location in relation to target tissue (e.g., vagus nerve 21), electrical stimulation therapy and/or sensing may be more consistent and/or have greater efficacy.

Endovascular device 16 (e.g., the elongated body of endovascular device 16) may configured to transform between a body delivery configuration (e.g., radially compressed configuration) and a body deployed configuration (e.g., radially expanded configuration). For example, when in the body deployed configuration, endovascular device 16 can define the one or more coiled portions. When in the body delivery configuration, endovascular device 16 can define a relatively lower cross-sectional profile (e.g., as measured in a radial direction) as compared to the body deployed configuration. For example, endovascular device 16 may not define the one or more coiled portions when in the delivery configuration. One or more portions of endovascular device 16, including the coiled portions of endovascular device 16, may be configured to be made straight or substantially straight in the body delivery configuration.

In some examples, endovascular device 16 defines multiple coiled portions. For instance, distal portion 15 of endovascular device 16 can define multiple coiled portions. In some examples, a more proximal portion of endovascular device 16 (e.g., a portion of endovascular device 16 proximal of distal portion 15) defines multiple coiled portions. In some examples, distal portion of endovascular device 16 defines a first coiled portion and a proximal portion of endovascular device 16 (e.g., a portion of endovascular device 16 proximal of distal portion 15) defines a second coiled portion. The first coiled portion can be configured to be positioned in a first blood vessel (e.g., jugular vein 13, as illustrated in FIG. 1). The first coiled portion can be proximate to (e.g., located in relatively close physical proximity to) expandable structure 19 and/or electrodes 17. The second coiled portion can be configured to be positioned in a second blood vessel (e.g., a subclavian vein, not shown in the example of FIG. 1). Having multiple coiled portions of the endovascular device 16 at different locations within the vasculature of patient 12 can help facilitate relatively better anchoring of endovascular device 16 as a whole within the vasculature of patient 12.

Endovascular device 16 includes any suitable medical instrument (e.g., device) configured to deliver electrical stimulation signals to tissue proximate electrodes 17. For example, endovascular device 16 can be and/or include one or more of a medical lead, a catheter, a guidewire, and/or another elongated body. Endovascular device 16 can include one or more elements configured to be electrically coupled to medical device 14 via an electrically conductive pathway (e.g., via one or more conductor wires) that runs between medical device 14 and electrodes 17. Endovascular device 16 has any suitable length that enables connection to medical device 14 either directly or indirectly, e.g., a length of 150 centimeters (cm) to 250 cm, such as 200 cm. Further, endovascular device 16 has a suitable length (e.g., as measured along a longitudinal axis of endovascular device 16) for accessing a target tissue site within the patient from a vascular access point. In examples in which endovascular device 16 accesses the jugular vein 13 and/or vasculature in a brain 18 of patient 12 from a femoral artery access point at the groin of the patient, endovascular device 16 has a length of about 100 cm to about 200 cm, although other lengths may be used. However, other access points may be used to introduce endovascular device 16 into vasculature of a patient, such as, but not limited to, a radial artery.

As used herein, “about” may indicate the exact value or nearly the exact value to the extent permitted by manufacturing tolerances. “About” can also refer to a certain percentage of the recited value (e.g., within about 1%, 5%, or 10%).

In some examples, endovascular device 16 is configured to be introduced in the vasculature of patient 12, such as to access jugular vein 13 and/or relatively more distal locations in a patient, such as the middle cerebral artery (MCA) in a brain of a patient.

Endovascular device 16 may include an elongated body that is structurally configured to be relatively flexible, pushable, and relatively kink- and buckle-resistant, so that it may resist buckling when a pushing force is applied to a relatively proximal portion to advance endovascular device 16 distally through vasculature, and so that it may resist kinking when traversing around a tight turn in the vasculature. Kinking and/or buckling of may hinder a clinician’s efforts to push the elongated body distally, e.g., past a turn. In some examples, endovascular device 16 includes one or more radiopaque components (e.g., platinum bands and/or other structures including platinum or platinum alloys) proximate electrodes 17 and/or expandable structure 19.

In some examples, endovascular device 16 can be navigated through vasculature (e.g., to jugular vein 13, brain 18, or other target tissue sites) with the aid of a guide member. The guide member can include an outer catheter, an inner catheter, a guide extension catheter, a guidewire, or the like or combination thereof.

In some examples, more than one of endovascular device 16 is introduced into, positioned in, and/or implanted within patient 12 to provide stimulation to and/or sense multiple anatomical regions, including one or more of both the left and right jugular veins, as well as in locations of brain 18. For example, two or more of endovascular device 16, which may be paired with one or more of medical device 14, may be configured of bilateral stimulation and/or sensing (e.g., of the left jugular vein and a right jugular vein). Endovascular device 16, including electrodes 17 and/or expandable structure 19, can be positioned in and/or implanted within a blood vessel for chronic therapy delivery and/or chronic sensing (e.g., on the order of months or even years) or for more temporary therapy delivery and/or sensing (e.g., on the order of days, such as less than a month or less than 6 months). Temporary therapy delivery may include one or more trial periods, such as to determine, evaluate, or confirm an efficacy of stimulation and/or sensing, and/or to select electrical stimulation parameters for chronic therapy delivery.

The electrical stimulation therapy described herein (e.g., VNS, DBS, or the like) may be used to treat various patient conditions, such as, a variety of illnesses including, but not limited to: reperfusion damage, cardiac ischemia, brain ischemia, stroke, traumatic brain injury, surgical or non-surgical acute kidney injury, inability of the intestine (bowel) to contract normally and move waste out of the body, postoperative ileus, postoperative cognitive decline or postoperative delirium, asthma, sepsis, bleeding control, myocardial infarction reduction, dysmotility, obesity, movement disorders, other neurodegenerative impairment, seizure disorders, psychiatric disorders (e.g., mood disorders). Treating any of these diseases may improve patient outcomes by shortening length of hospital stays and reducing medical costs.

The vasculature into which endovascular device 16 may be inserted and/or guided includes, but is not limited to, veins or arteries. For example, endovascular device 16 can be navigated from a vasculature access site (e.g., in the femoral artery, the radial artery, or another suitable access site) to one or more of a jugular vein (e.g., internal jugular vein and/or external jugular vein), a carotid artery (e.g., internal carotid artery, external carotid artery, and/or common carotid artery), as well as brain targets including the thalamostriate vein, the internal cerebral vein, the basal vein of Rosenthal, the inferior/superior sagittal sinus, the anterior choroidal artery, or any related combinations thereof.

A clinician can also select a particular blood vessel to position electrodes 17 within, such as to avoid certain regions to minimize or even eliminate adverse effects. For example, electrodes 17 can be oriented or positioned relative to vagus nerve 21 to avoid inadvertently providing electrical stimulation to anatomical regions (e.g., undesired anatomical regions) near the targeted anatomical region.

In some examples, endovascular device 16 is configured to be delivered to one or more target sites in vasculature of patient 12. Thus, rather than introducing endovascular device 16 into tissue in close proximity with vagus nerve 21 through an incision in the neck or chest area of patient 12, endovascular device 16 is configured to be navigated proximate to a target electrical stimulation site via vasculature of patient 12. The endovascular delivery of endovascular device 16 to target sites can help minimize the invasiveness of therapy system 10.

In some examples, one or more electrodes 17 are positioned on (e.g., mechanically coupled to, defined by, or otherwise carried by) expandable structure 19 of endovascular device 16, which is configured to expand radially outwards from a relatively low-profile (e.g., radially compressed) delivery configuration to a deployed (e.g., radially expanded) configuration. This may enable electrodes 17 to be held in apposition with a blood vessel wall, promote tissue ingrowth around electrodes 17 along the vessel wall (while still leaving a patent lumen to enable blood flow through the blood vessel, through expandable structure 19, despite implantation of endovascular device 16), which can reduce the overall power needed to deliver efficacious electrical stimulation therapy to a target tissue site, and help secure electrodes 17 in place in the blood vessel for chronic therapy delivery.

Medical device 14 can be an external medical device or an implantable medical device that includes electrical stimulation circuitry configured to generate and deliver electrical stimulation therapy to patient 12 and/or sensing circuitry configured to sense a patient parameter (e.g., a physiological signal) via one or more electrodes 17 of endovascular device 16. In the example of FIG. 1, endovascular device 16 is directly or indirectly mechanically and electrically coupled to medical device 14 via a header 11 of medical device 14. In some examples, header 11 defines a plurality of electrical contacts in one or more feedthrough portions (e.g., that are configured to electrically couple electrodes 17 to electrical stimulation generation circuitry and/or sensing circuitry within medical device 14).

In some examples, therapy system 10 includes one or more conductor wires (not shown in FIG. 1) extending between medical device 14 and electrodes 17. The one or more conductor wires can be configured to carry electrical signals between medical device 14 and electrodes 17 or vice versa. The conductor wires may extend along, be a part of, be incorporated into, and/or integrally formed as part of endovascular device 16. Other types of conductive elements for transmitting electrical signals (e.g., trace elements and the like) can additionally or alternatively be used. In some examples, header 11 includes multiple feedthrough portions, which may be respectively configured for receiving one of multiple portions of endovascular device 16. Header 11 may also be referred to as a connector block or connector of medical device 14. Endovascular device 16 may be mechanically coupled and/or electrically coupled to header 11 with the aid of a lead extension. However, in some examples, a lead extension is not used between header 11 and endovascular device 16, and endovascular device is directly mechanically and/or electrically connected to medical device 14 via header 11.

In some examples, medical device 14 is configured to be positioned in (e.g., implanted in) patient 12 in any suitable location, such as a location in a pectoral region. In other examples, medical device 14 is configured to be external to patient 12. Endovascular device 16 may be, for example, implanted within a vein (e.g., jugular vein 13) and one or more proximal wires/leads can remain within the venous system until they exit the venous system, such as through the subclavian vein in the chest or the internal jugular vein in the neck for implant in the pectoral region. In yet other examples, some or all of medical device 14 is configured to be implanted in the vasculature, e.g., as part of endovascular device 16.

As illustrated in FIG. 1, system 10 may also include a programmer 20, which may be a handheld device, portable computer, or workstation that provides a user interface to a user, for example a clinician or other user, such as a patient. The user may interact with the user interface to program electrical stimulation parameters for medical device 14.

With the aid of programmer 20 or another computing device, a clinician may select values for therapy parameters for controlling therapy delivery by therapy system 10. The values for the therapy parameters may be organized into a group of parameter values referred to as a “therapy program” or “therapy parameter set.” “Therapy program” and “therapy parameter set” are used interchangeably herein. In the case of electrical stimulation, the therapy parameters may include a combination of activated electrodes (also referred to herein as an electrode combination), a power, and an amplitude, which may be a current or voltage amplitude, and, if medical device 14 delivers electrical pulses, a pulse width, and a pulse rate for stimulation signals to be delivered to the patient. Other example therapy parameters include a slew rate, duty cycle, and phase of the electrical stimulation signal.

An electrode combination may include a selected subset of one or more electrodes 17 located on one or more of endovascular devices 16 mechanically coupled and/or electrically coupled to medical device 14. The electrode combination may also refer to the polarities of the electrodes in the selected subset. By selecting particular electrode combinations, a user may target particular tissue sites (e.g., anatomic structures) within patient 12. In addition, by selecting values for slew rate, duty cycle, phase amplitude, pulse width, and/or pulse rate, the user can attempt to generate an efficacious therapy for patient 12 that is delivered via the selected electrode subset.

Whether programmer 20 is configured for clinician or patient use, programmer 20 may be configured to communicate with medical device 14 or any other computing device via wireless or a wired communication. Programmer 20, for example, may communicate via wireless communication with medical device 14 using radio frequency (RF) telemetry techniques. Programmer 20 may also communicate with another programmer or computing device via a wired or wireless connection using any of a variety of local wireless communication techniques, such as RF communication according to the 802.11 or Bluetooth specification sets, infrared communication according to the Infrared Data Association (IRDA) specification set, or other standard or proprietary telemetry protocols. Programmer 20 may also communicate with another programming or computing device via a wired or wireless communication technique.

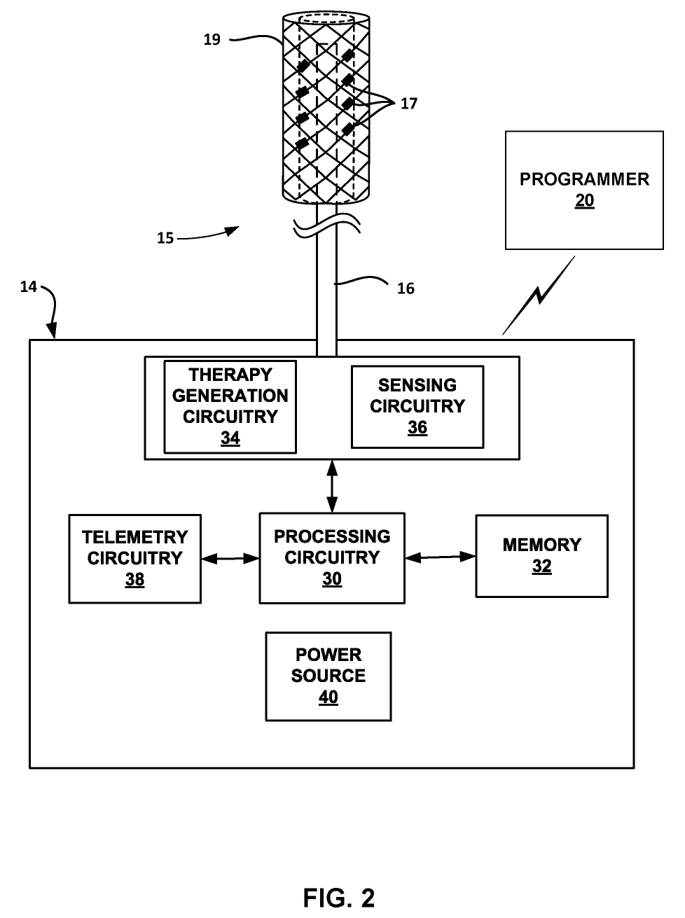

In some examples, in addition to or instead of delivering electrical stimulation to a target location (e.g., vagus nerve 21), medical device 14 or another device is configured to sense one or more patient parameters, such as bioelectric signals, either using electrodes 17 or other types of sensors that are carried by endovascular device 16. Bioelectric signals (also referred to herein as bioelectrical signals) can be sensed, and indications of sensed signals can be used by clinicians to make clinically relevant decision. In other examples, sense bioelectric signals are used as part of continuous feedback system in which medical device 14 adjusts one or more therapy parameter values based on sensed bioelectrical signals. Example bioelectric signals are described in further detail below with reference to FIG. 2.

In some examples, medical device 14 is configured to generate and deliver a suitable electrical stimulation signal, which can be a continuous time signal (e.g., a sinusoidal waveform or the like) or a plurality of pulses. In some examples, the electrical stimulation waveform generated by medical device 14 and delivered by one or more of electrodes 17 is a charge balanced, biphasic waveform. In some examples, such an electrical stimulation waveform consists of periodic pulses or otherwise include periodic pulses, or can include a continuous time waveform.

As noted above, in some examples, one or more electrodes 17 are positioned on expandable structure 19. In some examples, one or more sensors that are different from electrodes 17 are positioned on the same expandable structure (e.g., expandable structure 19) as one or more electrodes 17 or on a different expandable structure (e.g., a structure similar to or different from expandable structure 19) of endovascular device 16. Expandable structure 19 can have any suitable configuration that enables endovascular device 16 to assume a relatively low-profile configuration (also referred to herein as a “delivery” or “compressed” configuration in some examples) to facilitate delivery through vasculature to a target tissue site and expand radially outwards (relative to a central longitudinal axis of endovascular device 16) to position the one or more electrodes 17 closer to target tissue.

In some examples, expandable structure 19 is configured to expand radially outwards with sufficient force and to a cross-sectional dimension (e.g., a diameter) sufficient to position the one or more electrodes 17 in apposition with a blood vessel wall. Positioning one or more electrodes 17 in apposition with a blood vessel wall may help promote tissue ingrowth around electrodes 17, which can reduce the impedance and the overall power needed to deliver efficacious electrical stimulation therapy to a target tissue site, and help secure electrodes 17 in place in the blood vessel for chronic (e.g., on the order of months or even years) therapy delivery. Fixing endovascular device 16 in place within the blood vessel via the tissue ingrowth or, in some examples, using another fixation structures/anchoring mechanisms, such as tines, coils, barbs, or the like, can also help reduce the possibility of thrombosis.

Expandable structure 19 can be configured to expand radially outwards using any suitable technique and configuration. In some examples, expandable structure 19 includes a shape memory (e.g., nitinol) material that enables expandable structure 19 to assume a predetermined shape in the absence of a force (e.g., a compressive or tensile force) holding expandable structure 19 in a relatively low-profile delivery configuration. For example, expandable structure 19 can be configured to expand (e.g., self-expand) radially outwards upon deployment from an outer sheath (e.g., an outer catheter), or upon the proximal withdrawal of a straightening element (e.g., a guidewire or a mandrel) positioned in an inner lumen of the endovascular device 16. In some examples, expandable structure 19 is configured to expand radially outwards in response to proximal movement of a pull member attached to a distal portion of the endovascular device 16, in response to a distal movement of an elongated control member attached to the expandable structure, or with the aid of a balloon or the like.

Expandable structure 19 can have any suitable configuration in its deployed (e.g., expanded) configuration. In some examples herein, expandable structure 19 includes a plurality of interconnected struts to form a structure configured to expand radially outward (e.g., from a central longitudinal axis of expandable structure 19). For example, expandable structure 19 can include a tubular member, a basket, include one or more splines or arms configured to expand radially outwards, define one or more loops, define a helical or spiral element, or the like or combinations thereof, when in the deployed configuration. One or more expandable structures 19 may be disposed at various positions along endovascular device 16 (e.g., at one or more longitudinal positions along endovascular device 16). Expandable structure 19 can be formed from a plurality of structural elements (e.g., braided or mechanically coupled together) or can be a unitary structure (e.g., a laser cut nitinol tube). In some examples, expandable structure 19 is referred to herein as having a stent-like structure.

Expandable structure 19 can be mechanically coupled to a portion of endovascular device 16 (e.g., distal portion 15 of endovascular device 16). In some examples, expandable structure 19 is mechanically coupled to endovascular device 16 via a welded connection, a crimped connection, a bonded connection (e.g., via an adhesive and/or another suitable bonding agent), or another suitable mechanical connection.

In addition to, or instead of, chronic therapy delivery and/or chronic sensing, example devices, systems, and methods described herein can be used for more temporary applications. In some examples, a first endovascular device (e.g., configured like endovascular device 16 or having another configuration) is configured to be operated in an acute (e.g., temporary) trial mode for a trial period to determine, evaluate, or confirm an efficacy of stimulation and/or sensing. For example, endovascular device 16 (as well as electrodes 17, medical device 14, processing circuitry, etc.) may be configured to operate in the trial mode to determine the efficacy of one or more stimulation parameter values and/or one or more sensing parameters. After the acute trial period, the first endovascular device may be removed, and a second endovascular device (e.g., configured like endovascular device 16 or having another configuration) configured to operate in a chronic mode may be implanted for a chronic period for chronic (e.g., long term, or permanent) stimulation therapy or sensing. In some examples, a first endovascular device (e.g., for use in the acute trial mode) is configured to be implanted and subsequently removed after the trial period.

A trial period has a shorter intended duration as compared to a chronic period, though the ultimate length of the chronic period may be less than an intended duration due to one or more factors, such as a patient response that requires shortening the chronic period relative to the intended duration of the chronic period. In some examples, the trial period includes a trial period length on the order of minutes (e.g., 1 minute, 2 minutes, 3 minutes, 5 minutes, 30 minutes, 45 minutes, etc.), on the order of hours (e.g., 1 hour, 2 hours, 5 hours, 12 hours, etc.), on the order of days (e.g., 1 day, 2 days, 3 days, etc.), on the order of weeks (e.g., 1 week, 2 weeks, 3 weeks, etc.) on the order of months (e.g., 1 month, 2 months, 3 months, etc.), or longer. In some examples, one or more of endovascular devices may be used for multiple trial periods (e.g., successive trial periods) for determining an efficacy of one or more stimulation parameters and/or one or more sensing parameters.

Therapy system 10 may have any suitable configuration for delivering electrical stimulation to a target tissue site in patient 12 or sensing a patient parameter from an endovascular location (e.g., jugular vein 13). In some examples, therapy system 10 includes a first subset of electrodes of electrodes 17 configured for delivering electrical stimulation therapy and a second subset of electrodes of electrodes 17 configured to for sensing one or more patient parameters. In some examples, some or all electrodes of electrodes 17 are configured for both electrical stimulation therapy and for sensing one or more patient parameters. Therapy system 10 can include any suitable number of electrodes 17 and/or combination of different kinds of electrodes. In some examples, electrodes 17 include electrodes formed via one or more manufacturing processes. For example, electrodes 17 can include a first electrode type (e.g., an electrode configured for delivery of electrical stimulation therapy), a second electrode type (e.g., an electrode configured to sensing a signal), or any suitable combination thereof.

FIG. 2 is a functional block diagram illustrating components of an example medical device 14, which is configured to generate and deliver electrical stimulation therapy to patient 12 and, in some examples, sense one or more patient parameters, such as bioelectrical signals or other physiological parameter of patient 12. Medical device 14 includes processing circuitry 30, memory 32, therapy generation circuitry 34, sensing circuitry 36, telemetry circuitry 38, and power source 40.

Therapy generation circuitry 34 includes any suitable configuration (e.g., hardware) configured to generate and deliver electrical stimulation signals to target tissue (e.g., vagus nerve 21) in patient 12. Processing circuitry 30 is configured to control therapy generation circuitry 34 to generate and deliver electrical stimulation therapy via electrodes 17 of endovascular device 16. The therapy parameter values may be selected based on the patient condition being addressed, as well as the target tissue site in patient 12 for the electrical stimulation therapy. The electrical stimulation therapy can be provided via stimulation signals of any suitable form, such of stimulation pulses or continuous-time signals (e.g., sine waves).

Sensing circuitry 36 is configured to sense a physiological parameter of a patient. Sensing circuitry 36 may include any sensing hardware configured to sense a physiological parameter of a patient, such as, but not limited to, one or more electrodes, optical receivers, pressure sensors, or the like. The one or more sensing electrodes can be the same or different from electrodes 17 configured to deliver electrical stimulation therapy. In some examples, processing circuitry 30 stores the sensed physiological parameters in memory 32 or transmits the sensed parameters to another device via telemetry circuitry 38. In addition, in some examples, processing circuitry 30 can use the sensed physiological signals to control therapy delivery by therapy generation circuitry 34, e.g., the timing of the therapy delivery or one or more characteristics (e.g., parameters values) of the electrical simulation signal generated by therapy generation circuitry 34.

In some examples, sensing circuitry 36 is configured to sense a bioelectrical signal, which otherwise may be referred to as a patient parameter, via one or more electrodes 17 (e.g., all or a subset of electrodes 17). Thus, electrodes 17 can be configured to receive or transmit energy (e.g., current). In some examples, such as those in which electrodes 17 are placed proximate vagus nerve 21 (FIG. 1), example bioelectrical signals include muscle activation signals (e.g., laryngeal muscle activation), electrocardiogram (ECG), intracardiac electrogram (EGM), electromyogram (EMG). In other examples, such as those in which electrodes 17 are placed in or otherwise proximate brain 18, example bioelectrical signals include brain signals such as an EEG signal, an electrocorticogram (ECoG) signal, a signal generated from measured field potentials within one or more regions of brain 18, action potentials from single cells within brain 18 (referred to as “spikes”), or evoked potentials. Determining action potentials of single cells within brain 18 may require resolution of bioelectrical signals to the cellular level and provides fidelity for fine movements, i.e., a bioelectrical signal indicative of fine movements (e.g., slight movement of a finger).

In examples in which endovascular device 16 is configured to sense an evoked potential, endovascular device 16 may also be configured to generate a stimulus (e.g., via therapy generation circuitry 34, alone or in combination with processing circuitry 30) to elicit the evoked potential. For example, endovascular device 16 can generate and deliver electrical stimulation to tissue in brain 18 and sense an evoked compound action potential (ECAP). An ECAP is synchronous firing of a population of neurons which occurs in response to the application of a stimulus including, in some cases, an electrical stimulus by endovascular device 16. The ECAP may be detectable as being a separate event from the stimulus itself, and the ECAP may reveal characteristics of the effect of the stimulus on the tissue.

In some examples, sensing circuitry 36 and/or processing circuitry 30 includes signal processing circuitry configured to perform any suitable analog conditioning of the sensed physiological signals. For example, sensing circuitry 36 may communicate to processing circuitry 30 an unaltered (e.g., raw) signal. Processing circuitry 30 may be configured to modify a raw signal to a usable signal by, for example, filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof. In some examples, the conditioned analog signals are processed by an analog-to-digital converter of processing circuitry 30 or other component to convert the conditioned analog signals into digital signals. In some examples, processing circuitry 30 operates on the analog or digital form of the signals to separate out different components of the signals. In some examples, sensing circuitry 36 and/or processing circuitry 30 performs any suitable digital conditioning of the converted digital signals, such as low pass, high pass, band pass, notch, averaging, or any other suitable filtering, amplifying, performing an operation on the signal, performing any other suitable digital conditioning, or any combination thereof. Additionally or alternatively, sensing circuitry 36 may include signal processing circuitry to modify one or more raw signals and communicate to processing circuitry 30 one or more modified signals.

In some examples, processing circuitry 30, alone or in combination with therapy generation circuitry 34 and/or sensing circuitry 36, is configured to operate medical device 14 (including electrodes 17, endovascular device 16, etc.) in a trial mode for a trial period to determine an efficacy of electrical stimulation or sensing. As described above, a trial mode can include a trial period of stimulation and/or sensing to determine, evaluate, or confirm an efficacy of stimulation and/or sensing. In some examples, processing circuitry 30, alone or in combination with therapy generation circuitry 34 and/or sensing circuitry 36, is configured to deliver electrical stimulation therapy and/or sense a patient parameter during the trial period. In some examples, processing circuitry 30 is configured to determine, evaluate, or confirm an efficacy of stimulation and/or sensing. For example, processing circuitry 30 may determine one or more therapy parameters for chronic stimulation and/or sensing based on the trial period.

Although shown as part of medical device 14 in FIG. 2, in other examples, sensing circuitry 36 is part of a device separate from medical device 14. For example, sensing circuitry 36 can be part of an implantable sensing device implanted in patient 12.

Processing circuitry 30, as well as other processors, processing circuitry, controllers, control circuitry, and the like, described herein, may include any combination of integrated circuitry, discrete logic circuity, analog circuitry, such as one or more microprocessors, digital signal processors (DSPs), application specific integrated circuits (ASICs), or field-programmable gate arrays (FPGAs). In some examples, processing circuitry 30 includes multiple components, such as any combination of one or more microprocessors, one or more DSPs, one or more ASICs, or one or more FPGAs, as well as other discrete or integrated logic circuitry, and/or analog circuitry.

Memory 32 is configured to store program instructions, such as software, which may include one or more program modules, which are executable by processing circuitry 30. When executed by processing circuitry 30, such program instructions may cause processing circuitry 30 to provide the functionality ascribed to processing circuitry 30 herein. The program instructions may be embodied in software and/or firmware. Memory 32 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Processing circuitry 30 is configured to control telemetry circuitry 38 to send and receive information. Telemetry circuitry 38, as well as telemetry modules in other devices described herein, such as programmer 20 (FIG. 1), may accomplish communication by any suitable communication techniques, such as RF communication techniques. In addition, telemetry circuitry 38 may communicate with external medical device programmer 20 via proximal inductive interaction of medical device 14 with programmer 20. Accordingly, telemetry circuitry 38 may send information to external programmer 20 on a continuous basis, at periodic intervals, or upon request from medical device 14 or programmer 20.

Power source 40 is configured to deliver operating power to various components of medical device 14. Power source 40 may include a small rechargeable or non-rechargeable battery and a power generation circuit to produce the operating power. Recharging may be accomplished through proximal inductive interaction between an external charger and an inductive charging coil within medical device 14. In some examples, power requirements may be small enough to allow medical device 14 to utilize patient motion and implement a kinetic energy-scavenging device to trickle charge a rechargeable battery. In other examples, traditional batteries may be used for a limited period of time.

In some examples, endovascular device 16 is configured to be a standalone electrical stimulation device and can include one or more elements of medical device 14 illustrated in FIG. 2.

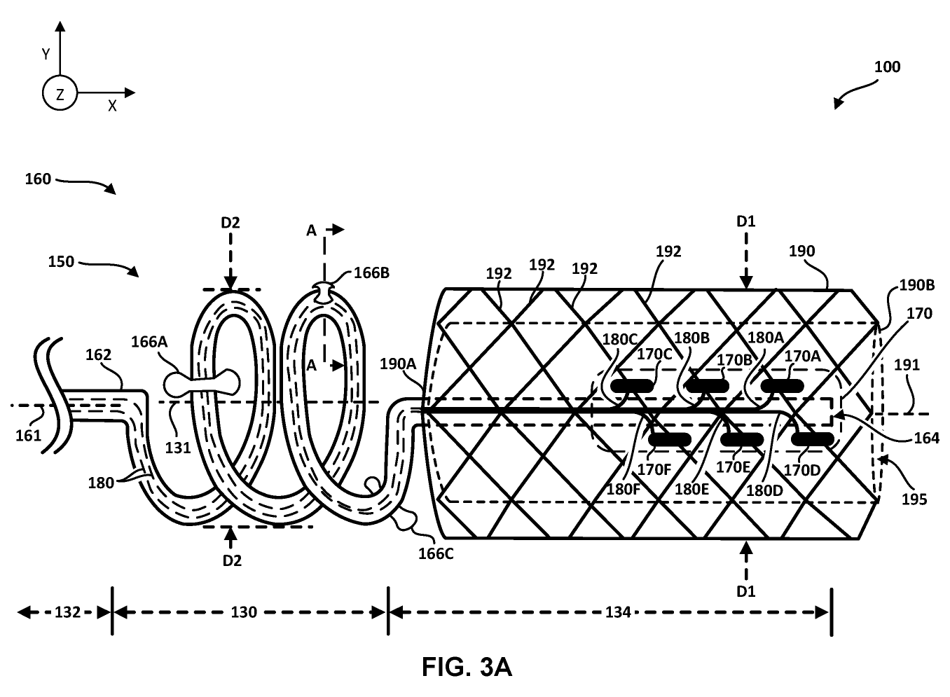

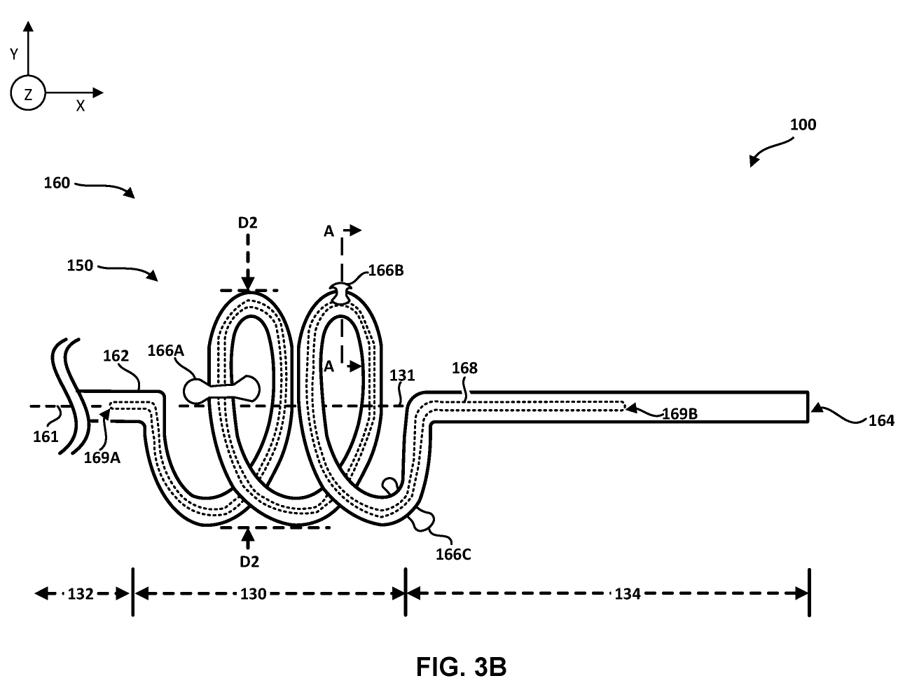

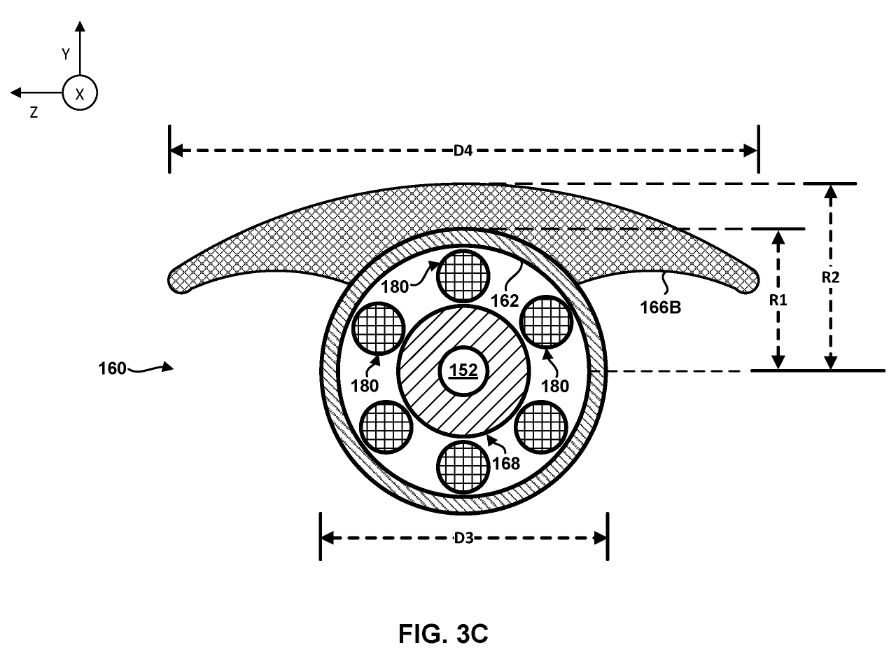

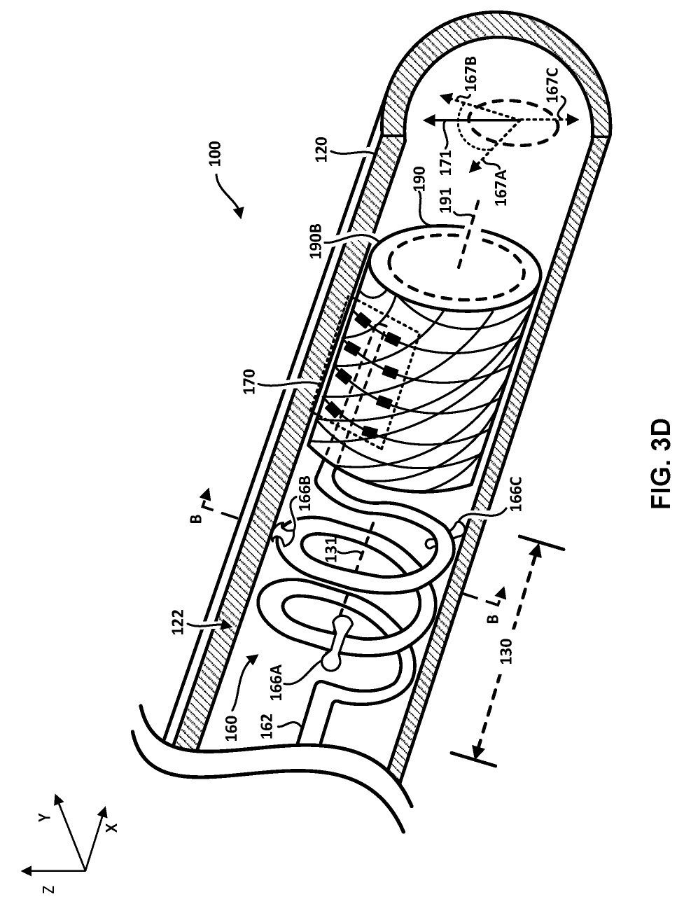

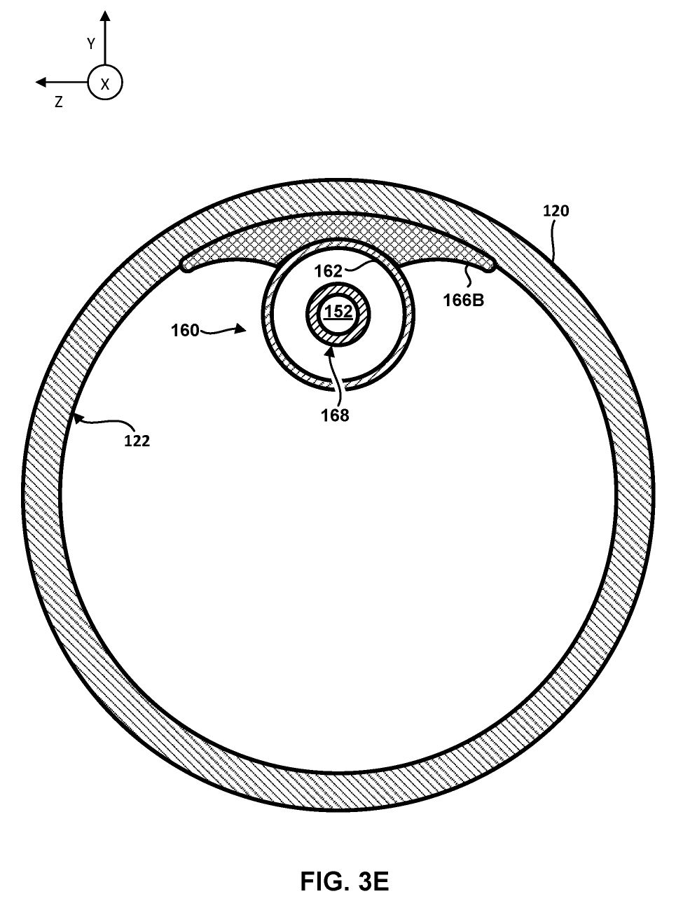

FIG. 3A illustrates an example endovascular therapy system 100, which is an example of therapy system 10 of FIG. 1. FIG. 3A illustrates a side view of endovascular therapy system 100. Endovascular therapy system 100 includes an endovascular device 160 and an expandable structure 190 at a distal portion 150 of endovascular device 160. As illustrated, endovascular therapy system 100 includes electrode 170A, electrode 170B, electrode 170C, electrode 170D, electrode 170E, and electrode 170F, collectively referred to herein and/or shown as electrodes 170. Endovascular device 160, expandable structure 190, distal portion 150, and electrodes 170 are examples of endovascular device 16, expandable structure 19, distal portion 15, and electrodes 17 as illustrated in and described with respect to FIG. 1, respectively.

FIG. 3B illustrates example endovascular therapy system 100 of FIG. 3A, but with certain features omitted for illustrative purposes. FIG. 3C illustrates a cross-sectional view of a portion of endovascular therapy system 100 of FIG. 3A and FIG. 3B. In the example of FIG. 3C, the cross-section is taken through the A-A section lines of FIG. 3A and FIG. 3B and facing in the positive x-axis direction according to the orthogonal x-y-z axes of FIG. 3A and FIG. 3B.

FIG. 3D illustrates endovascular therapy system 100 of FIG. 3A positioned within a blood vessel 120 having a blood vessel wall 122. Blood vessel 120 may be an example of jugular vein 13 of FIG. 1, and/or another suitable blood vessel into which endovascular therapy system 100 can be positioned. As illustrated in the example of FIG. 3D, expandable structure 190 is in the deployed configuration such that electrodes 170 are in apposition with and/or contacting blood vessel wall 122.

FIG. 3E illustrates a cross-sectional view of a portion of endovascular therapy system 100 within blood vessel 120 as illustrated in FIG. 3D. In FIG. 3E, the cross-section is taken through the B-B section lines of FIG. 3D and facing in the positive x-axis direction according to the orthogonal x-y-z axes of FIG. 3D.

Endovascular device 160 can have any suitable configuration, and may be configured according to the description of endovascular device 16 of FIG. 1. In some examples, and with reference to at least FIG. 3A and FIG. 3C, endovascular device 160 includes an elongated body 162 (e.g., a tubular body defining a lumen). With reference to FIG. 3A elongated body 162 of endovascular device 160 can extend between an elongated body proximal end (not shown in the examples of FIG. 3A) and an elongated body distal end 164. Elongated body distal end 164 may be a distalmost end of elongated body 162. In some examples, elongated body 162 of endovascular device 160 defines an elongated body central longitudinal axis 161 extending along endovascular device 160. Elongated body central longitudinal axis 161 may be a central longitudinal axis of elongated body 162.

In some examples, endovascular device 160 (e.g., elongated body 162 of endovascular device 160), which may be and/or include a medical lead, includes a suitable biocompatible polymer material. For example, of elongated body 162 of endovascular device 160 can include a thermoplastic material, such as Polycarbonate Urethane (PCU). In some examples, endovascular device 160 can additionally or alternatively include one or more of Polyurethane (PUR or PU), Polyethylene (PE), Polypropylene (PP), Polyetheretherketone (PEEK), Polyphenylsulfone (PPSU or PPSF), Polypropylene (PP), Nylon, Polyester, Polyethylene Terephthalate (PET), Polymethyl Methacrylate (PMMA), Polysulfone (PSU), and/or another suitable material.

In some examples, endovascular device 160 is configured to be at least partially introduced into, positioned in, and/or implanted within vasculature (e.g., blood vessel 120 as illustrated in FIG. 3D) of patient 12. Endovascular device 160 (e.g., elongated body 162) may include an electrically insulative material covering at least some portions of endovascular device 160 (e.g., one of the materials listed above). The electrically insulative material covering at least some portions of endovascular device 160 can electrically insulate elements disposed within elongated body 162 of endovascular device 160 (e.g., electrically insulate electrically conductive components, such as conductor wires 180, from blood or other tissue, such as when endovascular device 160 is positioned in or advanced through a blood vessel of patient 12).

In the example of FIG. 3A, endovascular therapy system 100 includes a plurality of conductor wires 180 (shown individually as conductor wire 180A, conductor wire 180B, conductor wire 180C, conductor wire 180D, conductor wire 180E, and conductor wire 180F, but collectively referred to and/or shown as conductor wires 180). Conductor wires 180 can be configured to electrically connect electrodes 170 to a medical device (e.g., medical device 14 of FIG. 1). Each of conductor wires 180A–180F can extend along (e.g., within) at least a portion of endovascular device 160 (e.g., within at least a portion of elongated body 162). In some examples, conductor wires 180 form a multi-filar coil within at least a portion of endovascular device 160 (e.g., within elongated body 162 of endovascular device 160). In some examples, some or all of conductor wires 180 are part of endovascular device 160, while in other examples, some or all of conductor wires 180 are separate components from endovascular device 160.

In some examples, at least a portion of each of conductor wires 180A–180F are housed by the insulative material of endovascular device 160 (e.g., the insulative material of elongated body 162 of endovascular device 160). For example, each of conductor wires 180A–180F can extend within a lumen of endovascular device 160 (e.g., with elongated body 162 of endovascular device 160, as illustrated in FIG. 3C). In some examples, insulative material of endovascular device 160 (e.g., of elongated body 162) can be configured to electrically insulate portions of conductor wires 180 that run along the length of endovascular device 160. As illustrated in the example of FIG. 3A, each of conductor wires 180A–180F can extend through and/or outward from elongated body 162, such as to branch out to mechanically connect and/or electrically connect to one or more of electrodes 170. In some examples, each of conductor wires 180A–180F extends along at least a portion of expandable structure 190.

Some or all of conductor wires 180 may include a material or combination of materials configured to facilitate relatively high flexibility, high axial extensibility, and/or high fatigue resistance. For example, one or more wires of conductor wires 180 includes a beta-titanium alloy. In some examples, the beta-titanium alloy comprises a Ti-15Mo alloy. Certain beta-titanium alloys, including Ti-15Mo alloy and similar titanium alloys enable higher wire count coils (e.g., twelve wire or greater, including equal to or greater than sixteen wire coils), such as for situations in which a relatively high number of individually controlled electrodes are needed in a small space including nerve stimulation and/or sensing from endovascular locations.

In some examples, each of conductor wires 180A–180F are electrically connected to a respective electrode of electrodes 170A–170F. In some examples, each of electrodes 170A–170F is configured to receive and/or otherwise mechanically couple to one or more conductor wires of conductor wires 180A–180F (e.g., to facilitate the electrical connection between each of conductor wires 180A–180F and one or more of electrodes 170). For example, each electrodes 170A–170F can define one or more conductor holes configured to receive one or more conductor wires 180A–180F, such as for electrically coupling respective electrodes to a medical device (e.g., medical device 14 of FIG. 1). Electrodes 170 can include an electrical contact portion configured to facilitate electrical connection to conductor wires 180.

In some examples, more than one of electrodes 170 are electrically connected to a common conductor wire of conductor wires 180 (e.g., some of electrodes 170 can be “shorted” together). For example, one of conductor wires 180A–180F can be configured to connect to a least a first electrode and a second electrode of electrodes 170 (e.g., such that a medical device 14 can simultaneously control each of the first electrode and the second electrode of electrodes 170 together). Shorting of at least some of electrodes 170 can facilitate control of multiple electrodes at the same time (e.g., for delivery of electrical stimulation therapy and/or sensing).

As illustrated in at least FIGS. 3A and FIG. 3D, electrodes 170 are carried by expandable structure 190. In some examples, expandable structure 190 is configured to position and/or orient electrodes 170 within vasculature of a patient (e.g., patient 12 of FIG. 1). In some examples, at least some of electrodes 170 are carried by and/or mechanically connected to struts 192 of expandable structure 190. In some examples, electrodes 170 are carried by and/or disposed on expandable structure 190, and expandable structure 190 is configured to transform from a relatively low-profile delivery configuration to a deployed configuration in a blood vessel of a patient (e.g., within jugular vein 13 of patient 12 as discussed in relation to FIG. 1 and/or within a cranial blood vessel of patient 12).

Expandable structure 190 is an example of expandable structure 19 as discussed in connection with FIG. 1, and can include any suitable shape and materials. Expandable structure 190 can have any suitable configuration for positioning electrodes 170 for delivering stimulation therapy and/or sensing one or more patient parameters of patient 12 from an endovascular location. In some examples, as illustrated in the example of FIG. 3A, expandable structure 190 includes a body portion (e.g., a cylindrical body portion) extending between expandable structure proximal end 190A and an expandable structure distal end 190B. In some examples, as illustrated in the example of FIG. 3A, expandable structure 190 includes a plurality of interconnected struts 192. In some examples, expandable structure 190 defines a tubular structure defining an expandable structure lumen 195. In some examples, elongated body 162 of endovascular device 160 is mechanically coupled to expandable structure 190 such that at least a portion of elongate body 162 (e.g., at least elongated body distal end 164) is positioned within expandable structure lumen 195. In some examples, struts 192 are interconnected to form the tubular (e.g., stent-like) structure. In some examples, expandable structure 190 defines a central longitudinal axis 191.

In some examples, expandable structure 190 is configured to expand (e.g., self-expand and/or via an expansion mechanism such as a balloon) radially outward from central longitudinal axis 191 to a deployed configuration. Such expansion can enable expandable structure 190 to position electrodes 170 into apposition with a blood vessel wall (e.g., for delivering electrical stimulation therapy to tissue of patient 12 proximate the blood vessel and/or sensing a patient parameter from a location within the blood vessel). Expandable structure 190 can include one or more of a self-expanding structure, including one or more of a self-expanding stent and/or another suitable expandable structure that includes one or more struts as described herein.

In the example of FIG. 3A, expandable structure 190 defines a greatest cross-sectional dimension D1 (e.g., when expandable structure 190 is in the deployed configuration). In some examples, dimension D1 is a diameter (e.g., in examples in which expandable structure 190 defines a circular or substantially circular cross-section). In some examples, dimension D1 is selected such that expandable structure 190 and/or electrodes 170 are positioned into apposition with a blood vessel wall (e.g., blood vessel wall 122 of blood vessel of 120, as illustrated in FIG. 3D) when expandable structure 190 is in the deployed configuration.

Expandable structure 190 can include suitable configurations for mechanically coupling to and/or carrying one or more electrodes of electrodes 170. In some examples, expandable structure 190 includes structural features configured to facilitate mechanical coupling of electrodes 170 to expandable structure 190, as well as orient electrodes 170 with respect to expandable structure 190. In some examples, one or more of struts 192 are configured to mechanically couple to one or more electrodes 170. In addition to or instead of struts 192, in some examples, expandable structure 190 includes other structures (e.g., weld pads, projections, or other structural features) configured to receive, mechanically to, or otherwise carry one or more of electrodes 170. Expandable structure 190 can also be configured to orient electrodes 170 to face radially outward from central longitudinal axis 191 (e.g., when expandable structure 190 is in the deployed configuration). In some examples, expandable structure 190 is configured to position electrodes 170 in apposition with a blood vessel wall (e.g., after therapy system 100 including expandable structure 190 is advanced proximate to a target location in the vasculature of a patient, such as patient 12 of FIG. 1).

Endovascular therapy system 100, including expandable structure 190, may be configured to have a relatively low-profile configuration to facilitate delivery and/or placement into relatively narrow and/or tortuous vessels. Once proximate a target location, expandable structure 190 can be configured to transform to the deployed configuration (e.g., to position the one or more of electrodes 170 to deliver electrical stimulation to tissue of patient 12 or sense a patient parameter from a location within the blood vessel). In the deployed configuration, expandable structure 190, which can include interconnected struts 192, is expanded radially outward (e.g., relative to central longitudinal axis 191 of expandable structure 190) as compared to the relatively low-profile configuration of expandable structure 190. In some examples, when expandable structure 190 is in a deployed configuration, electrodes 170 are flush or nearly flush with an outer surface of expandable structure 190. In some examples, expandable structure 190 is configured to position electrodes 170 such that at least one surface of each of electrodes 170 is flush or nearly flush with the outer surface of expandable structure 190 (e.g., when expandable structure 190 is in the deployed configuration).

Endovascular therapy system 100 can include any suitable number of electrodes 170 for delivery of stimulation therapy (e.g., electrical stimulation therapy) and/or sensing from an endovascular location. While the example of FIG. 3A illustrates endovascular therapy system as including six of electrodes 170, endovascular therapy system 100 can include any suitable number of electrodes 170 (e.g., one electrode, two electrodes, three electrodes, four electrodes, five electrodes, six electrodes, seven electrodes, eight electrodes, nine electrodes, ten electrodes, twelve electrodes, fifteen electrodes, twenty electrodes, thirty electrodes, or more). Each of electrodes 170 can be disposed at respective spaced-apart locations along and/or around expandable structure 190.

Expandable structure 190 can include electrodes 170 at multiple circumferential positions around expandable structure 190 and/or multiple longitudinal positions along expandable structure 190 (e.g., spaced apart along central longitudinal axis 191). Longitudinal spacing and/or circumferential spacing between adjacent electrodes 170 can correspond to desired longitudinal spacing and/or circumferential spacing between adjacent electrodes 170 for therapeutically effective endovascular stimulation and/or sensing. In some examples, circumferential spacing between adjacent electrodes (e.g., once mechanically coupled to expandable structure 190 and with no other intervening electrodes) is about 4 millimeters (mm) to about 8 mm around expandable structure 190. In some examples, an axial spacing and/or longitudinal spacing between adjacent electrodes 170 is about 1 mm to about 10 mm, such as about 5 mm to about 10 mm. In some examples, a circumferential spacing between adjacent electrodes 170 is less than an axial spacing and/or longitudinal spacing between adjacent electrodes 170. Although referred to as circumferential positions, in some examples, expandable structure 190 is not circular in cross-section (the cross-section being taken in a direction orthogonal to central longitudinal axis 191). In such examples, the circumferential positions may still refer to the rotational position about central longitudinal axis 191.

In some examples, electrodes 170 can form an electrode array. In some examples, each of electrodes 170 in the electrode array generally face outward in a common radial direction (e.g., in a common radial direction outward from central longitudinal axis 191 of expandable structure 190). Expandable structure 190 can be configured to be rotated (e.g., within the vasculature of patient 12 as described with respect to FIG. 1) to position the electrode array (e.g., including electrodes) 170 to face toward a target location and/or anatomical structure (e.g., toward vagus nerve 21 in the example of FIG. 1).

Electrodes 170 can be fabricated using any suitable method. In some examples, electrodes 170 are formed from a suitable machining (milling, turning, grinding, or electrical discharge machining) and/or stamping process. Electrodes 170 can be formed separately from, and subsequently mechanically coupled to, expandable structure 190. In other examples, electrodes 170 are integrally formed with expandable structure 190.

In some examples, one or more elements of therapy system 100 are configured to facilitate positioning of electrodes 170 at the target site (e.g., via radiographic and/or radiopaque portions that indicate a positioning of electrodes 170). For example, therapy system 100 can include a radiographic or radiopaque marker to indicate an axial position and/or circumferential position of one or more of electrodes 170. In some examples, at least a portion of therapy system 100 is aligned with one or more of electrodes 170 (and/or circumferentially aligned an array of electrodes formed from a group of electrodes 170) to indicate a direction (e.g., a radial direction) faced by electrodes 170. In some examples, therapy system 100 (e.g., one or more components of therapy system 100) includes a radiographic marker that is circumferentially aligned with one or more of electrodes 170 and/or an array of electrodes 170. For example, one or more of elongated body 162 of and/or expandable structure 190 of endovascular device 160 (or sub-components thereof) includes a radiographic or radiopaque material circumferentially aligned with electrodes 170 and configured to indicate a radial direction (e.g., a radial direction outwards from central longitudinal axis 191) faced by electrodes 170 (e.g., when expandable structure 190 is in the deployed configuration).

In some examples, one or more of struts 192 of expandable structure 190 is directly mechanically coupled to elongated body 162 of endovascular device 160. In some examples, elongated body 162 of endovascular device 160 is directly mechanically coupled to expandable structure 190 such that elongated body distal end 164 (e.g., a distalmost end of elongated body 162) is positioned distal to a proximal-most electrode of electrodes 170 (e.g., electrode 170C of FIG. 3A). In some examples, elongated body 162 is directly mechanically coupled to expandable structure 190 such that elongated body distal end 164 (e.g., a distalmost end of elongated body 162) is positioned distal to a distal-most electrode of electrodes 170 (e.g., electrode 170D FIG. 3A). In some examples, elongated body 162 is directly mechanically coupled to expandable structure 190 such that elongated body distal end 164 (e.g., a distalmost end of elongated body 162) is positioned distal to a proximal-most electrode of electrodes 170 (e.g., electrode 170C of FIG. 3A) but proximal to a distal-most electrode of electrodes 170 (e.g., electrode 170D FIG. 3A).

Although FIG. 3A is described with respect to electrodes 170 that are configured to deliver electrical stimulation therapy and/or sense electrical signals, endovascular therapy system 100 can additionally or alternatively include other types of therapy delivery elements and/or sensors. In some examples, endovascular therapy system 100 includes one or more ultrasound transducers, chemical delivery elements (e.g., fluid delivery elements and/or drug elution elements) which can be configured to be attached to expandable structure 190 using a similar method of attachment as electrodes 170. In some examples, endovascular therapy system 100 additionally or alternatively includes one or more temperature sensors, pressure sensors, optical sensors, impedance sensors, chemical sensors, and/or other suitable types of sensors, which can be configured to be attached to expandable structure 190 using a similar method of attachment as electrodes 170.

In some examples, endovascular device 160 includes and/or defines one or more structural features that enable the endovascular device 160 to absorb loads and/or forces while minimizing or even eliminating the transfer of such loads and/or forces to expandable structure 190. Such minimization and/or elimination of loads and/or forces to expandable structure 190 can, in turn, limit undesirable movement of the expandable structure 190 and/or electrodes 170. As used herein, the terms “force” and “load” can be used interchangeably, and generally refer to a push or pull exerted on an object, resulting from an interaction with another object, which can cause a change in the object’s motion, speed, or direction.

In some examples, endovascular device 160 (e.g., elongated body 162 of endovascular device 160) includes and/or defines a coiled portion 130. In some examples, as illustrated in the example of FIG. 3A, coiled portion 130 is a portion of elongated body 162 formed into a coil (e.g., a coil shape having multiple turns). The coil shape of coiled portion 130 can include multiple turns of elongated body 162 defining a pitch (e.g., spacing) between adjacent turns (e.g., as measured in the x-axis direction according to the orthogonal x-y-z axes of FIG. 3A). The pitch (e.g., spacing between adjacent coil turns) of coiled portion 130 can be about 0.5 mm to about 10 mm and/or any value therebetween. In some examples, coiled portion 130 has a constant pitch or substantially constant pitch (e.g., equal or substantially equal spacing between most or all adjacent coil turns).

In some examples, coiled portion 130 is configured to absorb and/or accommodate axial forces (e.g., forces that tend to push or pull on at least a portion of endovascular device 160, such as on elongated body 162), such that the transfer of such forces to expandable structure 190 is reduced or even eliminated. For example, coiled portion 130 may be configured to absorb and/or accommodate loads of up to 0.5 pounds without transferring such load to expandable structure 190. Coiled portion 130 can be configured to absorb and/or accommodate greater loads (e.g., greater than 0.5 pounds). Additionally or alternatively, coiled portion 130 can be configured to accommodate rotational forces and/or twisting forces (e.g., forces that tend cause a portion of endovascular device 160, such as elongated body 162, to rotate or twist), such that the transfer of such forces to expandable structure 190 is reduced or even eliminated. Such minimization and/or elimination of forces to expandable structure 190 can, in turn, limit undesirable movement of the expandable structure and/or the electrodes.

In some examples, coiled portion 130 is configured to limit or prevent a force (e.g., an axial force, rotational force, twisting force, and/or the like) applied to endovascular device 160 (e.g., to elongated body 162 of endovascular device 160) from being transferred to expandable structure 190 and/or the electrodes 170 (e.g., when expandable structure 190 and/or elongated body 162 are positioned within vasculature of a patient). Such forces can be the result of various physiological functions of a patient, such as from movement of a patient (e.g., normal movement, such as from breathing, movement such as turning of the head, standing up, sitting down, and/or the like). Additionally or alternatively, such forces applied to elongated body 162 that would otherwise cause movement of expandable structure 190 can result from forces placed on elongated body 162 during a surgical procedure in which elongated body 162 is placed within patient 12 (e.g., due to retraction of a delivery catheter relative to elongated body 162, due to connecting a proximal end of endovascular device 160 to a medical device, and/or the like). For example, in some examples, coiled portion 130 can at least partially deform when an axial force (e.g., a force that would tend to lengthen coiled portion 130 or a force that would tend to compress coiled portion is applied to coiled portion 130). In this way, coiled portion 130 can be configured to “absorb” forces and/or loads that would otherwise be transferred to expandable structure 190 by virtue of the mechanical connection between elongated body 162 and expandable structure 190.

In some examples, coiled portion 130 of endovascular device 160 is configured to absorb forces applied to elongated body 162 of endovascular device 160, e.g., axial forces, rotational forces, and/or twisting forces, while minimizing or even eliminating such forces from being transferred to expandable structure 190. With reference to FIG. 3D, this minimization and/or elimination of forces to expandable structure 190 can limit and/or prevent movement of electrodes 170 relative to target tissue radially outside of blood vessel 120. Such minimization and/or elimination of forces prior to adequate endothelialization and/or in the absence of adequate endothelialization can ensure that expandable structure 190 and/or the electrodes 170 remain in a relatively stable location. In some examples, at least a portion of coiled portion 130 of elongated body 162 of endovascular device 160 is configured to deform (e.g., extend, compress, and/or the like) without causing expandable structure 190 and/or electrodes 170 to move (e.g., axially translate, rotate, and/or the like).

Coiled portion 130 can have any suitable configuration. In some examples, coiled portions 130 forms a plurality of coil loops. As illustrated in at least FIG. 3A, coiled portion 130 (e.g., including the plurality of coil loops) can define a central longitudinal coil axis 131 extending through a radial center of coiled portion 130. Coiled portion 130 can define any suitable number of coil loops (e.g., a least one coil loop, at least two coil loops, at least three coil loops, or more). In some examples, coiled portion 130 defines two coil loops to ten coil loops. Each coil loop of coiled portion 130 can extend around central longitudinal coil axis 131. Having multiple (e.g., two or more) loops can enable coiled portion 130 to extend and/or compress to a greater extent without causing movement of expandable structure 190 and/or electrodes 170. In such examples, a majority of elongated body 162 of endovascular device 160 (e.g., more than 90 percent) is straight or substantially straight, and does not define any coiled portions.

Coiled portion 130 can have any suitable positioning relative to other components of endovascular therapy system 100. In some examples, as illustrated in the example of FIG. 3A, coiled portion 130 is located and/or positioned proximal of (e.g., entirely proximal to) expandable structure 190 and electrodes 170. Such positioning of coiled portion 130 relative to expandable structure 190 can ensure that coiled portion does not interfere with (e.g., physically interfere with, such as by touching) expandable structure 190. In some examples, however, coiled portion 130 is relatively close to expandable structure 190 (e.g., within about 10 cm, such as within about 5 cm or less), such as to reduce the likelihood of forces and/or loads acting on a substantially straight portion endovascular device 160 (e.g., elongated body 162) between coiled portion 130 and expandable structure 190 that could cause expandable structure 190 to move.

In some examples, as illustrated in the example of FIG. 3A, coiled portion 130 of endovascular device 160 does not include any electrodes. In some examples, all electrodes of endovascular therapy system 100 are positioned on expandable structure 190. In some examples, as illustrated in FIG. 3A, all electrodes 170 of endovascular therapy system 100 are distal to coiled portion 130 and/or any other coiled portions of endovascular device 160 in examples in which endovascular device 160 has more than one coiled portion 130.

In some examples, as illustrated in at least the examples of FIG. 3A and FIG. 3B, elongated body 162 of endovascular device 160 (e.g., at distal portion 150 of endovascular device 160) includes a distal segment 134. Distal segment 134 of endovascular device 160 can extend distally of coiled portion 130 and be configured to mechanically couple to a portion of expandable structure 190. Distal segment 134 of endovascular device 160 can be straight or substantially straight. In some examples, elongated body central longitudinal axis 161 of distal segment 134 and central longitudinal axis 191 of expandable structure 190 are aligned and/or parallel). Such configuration(s) of endovascular device 160 can enable a relatively short distance between endovascular device 160 and electrodes 170, such as to reduce a distance that each of conductor wires 180 extends out from endovascular device 160 (e.g., from elongated body 162 of endovascular device 160) to mechanically and/or electrically couple to each of electrodes 170. Reducing the distance that conductor wires 180 extend out from endovascular device 160 can facilitate reduced fatigue on conductor wires 180, which can extend the longevity and/or maintain the functionality of conductor wires 180.

In some examples, as illustrated in at least the examples of FIG. 3A and FIG. 3B, elongated body 162 of endovascular device 160 (e.g., at distal portion 150 of endovascular device 160) includes a proximal segment 132. Proximal segment 132 of endovascular device 160 can extend proximally of coiled portion 130. Proximal segment 132 can be substantially straight (e.g., as compared to coiled portion 130). Proximal segment 132 can extend to a proximal end (e.g., a proximalmost end) of endovascular device 160.

Endovascular device 160 (e.g., elongated body 162 of endovascular device 160) may configured to transform between a body delivery configuration and a body deployed configuration. The configuration (e.g., shape) of endovascular device 160 illustrated in FIG. 3A may be an example of endovascular device 160 in the body deployed configuration. In some examples, a portion of endovascular device 160 (e.g., a portion of elongated body 162) defines coiled portion 130 in the body deployed configuration. For example, as illustrated in the example of FIG. 3A, wherein in the body deployed configuration, distal portion 150 of endovascular device 160 defines coiled portion 130.

In some examples, endovascular device 160 (e.g., elongated body 162 of endovascular device 160) defines a relatively lower profile (e.g., as measured in a radial direction, such as relative to elongated body central longitudinal axis 161 and/or central longitudinal coil axis 131) in the body delivery configuration as compared to the body deployed configuration. In some examples, endovascular device 160 (e.g., elongated body 162 of endovascular device 160) does not define coiled portion 130 in the body delivery configuration. In some examples, endovascular device 160 (e.g., elongated body 162 of endovascular device) does define coiled portion 130 in the body delivery configuration, but coiled portion 130 can have a reduced cross-sectional dimension (e.g., a reduced diameter).

Coiled portion 130 can define any suitable shape and/or size. In some examples, as illustrated in the example of FIG. 3A, coiled portion 130 defines a greatest cross-sectional dimension D2. In some examples, dimension D2 is a diameter (e.g., in examples in which coiled portion 130 defines a circular or substantially circular cross-section). In some examples, coiled portion 130 defines dimension D2 when endovascular device 160 (e.g., elongated body 162 of endovascular device 160), including coiled portion 130, is in the body deployed configuration. In some examples, elongated body 162 has a pre-formed shape such that endovascular device 160 is configured to deploy to and/or return to a configuration in which endovascular device 160 defines coiled portion 130 having greatest cross-sectional dimension D2 (e.g., in the absence of external forces tending to hold endovascular device 160 in a different shape).

In some examples, greatest cross-sectional dimension D2 is selected such that coiled portion 130 contacts or nearly contacts a blood vessel wall of a blood vessel in which coiled portion 130 resides (e.g., blood vessel 120 of FIG. 3D). Such sizing can ensure that coiled portion 130 does not cause potentially undesirable disruption of blood flow in the blood vessel in which coiled portion 130 resides. Additionally or alternatively, such sizing of coiled portion 130 can reduce a likelihood of malapposition of electrodes 170 and/or expandable structure 190 against a blood vessel (e.g., blood vessel 120 of FIG. 3D).

In some examples, coiled portion 130 defines a similar or slightly smaller cross-sectional profile as compared to expandable structure 190 (e.g., when both of coiled portion 130 and expandable structure 190 are in respective expanded and/or deployed configurations). For example, in some examples, greatest cross-sectional dimension D2 of coiled portion 130 is similar to or slightly smaller than greatest cross-sectional dimension D1 of expandable structure 190. Greatest cross-sectional dimension D2 can be smaller than greatest cross-sectional dimension D1 by any suitable amount, such as about 1 percent to about 10 percent, and/or any value therebetween (e.g., 2 percent, 3 percent, 4 percent, 5 percent, 6 percent, 7 percent, 8 percent, 9 percent). Such relative sizing of coiled portion 130 and expandable structure 190 can ensure that coiled portion 130 does not affect the ability of expandable structure 190 and/or electrodes 170 to be brought into and to maintain apposition with a blood vessel wall (e.g., blood vessel wall 122 of blood vessel 120, as illustrated in FIG. 3D).

Endovascular device 160 (e.g., elongated body 162 of endovascular device) can include and/or define any suitable shape and/or form factor in each of body delivery configuration and the body deployed configuration. Elongated body 162 may define a relatively lower profile (e.g., radial profile) and/or form factor in the body delivery configuration (e.g., as compared to the body deployed configuration of elongated body 162). Coiled portion 130 may define a greatest cross-sectional dimension smaller than dimension D2 (e.g., as illustrated in at least FIG. 3A) in the body delivery configuration of endovascular device 160. For example, elongated body 162 of endovascular device 160 (e.g., including coiled portion 130) can be straight or substantially straight in the body delivery configuration (e.g., and may not define coiled portion 130).

In some examples, elongated body 162 of endovascular device 160, including coiled portion 130, can be configured to be straightened (e.g., collapsed) when advanced through a delivery catheter and/or sheath. In some examples, subsequent to being advanced from such a delivery catheter or sheath, elongated body 162 of endovascular device 160 is configured to self-deploy (e.g., expand, such as radially expand) from the body delivery configuration to the body deployed configuration. For example, elongated body 162 of endovascular device 160 can be configured to deploy to the body deployed configuration to define coiled portion 130 by advancing at least coiled portion 130 distally of a sheath and/or other elongated body surrounding coiled portion 130 (e.g., wherein the sheath and/or other elongated body holds elongated body 162, including coiled portion 130, in the body delivery configuration, which may be an uncoiled configuration and/or low-profile configuration). In some examples, elongated body 162 of endovascular device 160 is additionally or alternatively configured to define coiled portion 130 upon removal of a straightening element (e.g., a straightening wire within a lumen of endovascular device 160). In examples in which elongated body 162 of endovascular device 160 self-deploys to the body deployed configuration, endovascular device 160 defines coiled portion 130 once transformed to the body deployed configuration.

Coiled portion 130 can have other suitable configurations. In other examples, at least a portion of coiled portion 130 extends distally of expandable structure proximal end 190A. In some examples, at least a portion of coiled portion 130 extends within expandable structure lumen 195.

The example of FIG. 3B illustrates endovascular therapy system 100 of FIG. 3A, but with certain components omitted for illustrative purposes. In some examples, endovascular device 160 includes one or more structural features and/or components that enable coiled portion 130 to assume and/or maintain the coiled shape of coiled portion 130. In some examples, as illustrated in the example of FIG. 3B, endovascular device 160 (e.g., coiled portion 130 of endovascular device 160) includes a shaped rod 168. Shaped rod 168 can define a pre-formed coil shape. Shaped rod 168 can be configured to maintain the coil shape of coiled portion 130. Shaped rod 168, when positioned within and/or along endovascular device 160, can be configured to cause endovascular device 160 to assume and/or maintain a coil shape such that endovascular device 160 defines coiled portion 130. Shaped rod 168 can have a pre-configured number of coil loops having a pre-configured diameter and/or coil pitch (e.g., spacing between adjacent coil loops).

As illustrated in the example of FIG. 3B, shaped rod 168 extends along at least a portion of endovascular device 160. In some examples, coiled portion 130 includes shaped rod 168 positioned within (e.g., within a lumen of) endovascular device 160 (e.g., within a lumen of elongated body 162 of endovascular device 160). Shaped rod 168 can extend along (e.g., within) at least a portion of coiled portion 130 of endovascular device 160. In some examples, shaped rod 168 extends beyond (e.g., proximally and distally of) coiled portion 130 of endovascular device 160. The portions of shaped rod 168 extending beyond (e.g., proximally and distally of) coiled portion 130 of endovascular device 160 can define a shape different than a coil shape (e.g., such portions can straight or substantially straight). In the example of FIG. 3B, shaped rod 168 extends between a rod proximal end 169A and a rod distal end 169B. In some examples, at least a portion of shaped rod 168 (e.g., including rod proximal end 169A) extends into proximal segment 132 of endovascular device 160. In some examples, at least a portion of shaped rod 168 (e.g., including distal end 169B) extends into distal segment 134 of endovascular device 160. The portions of shaped rod 168 extending into either of proximal segment 132 and/or distal segment 134 (e.g., non-coiled portions of endovascular device 160) can increase the rigidity of these portions of endovascular device 160. The increased rigidity of proximal segment 132 and/or distal segment 134 can enable these portions of endovascular device 160 to maintain a desired shape and/or form factor (e.g., uncoiled and/or another shape), such as to enable endovascular device 160 to better conform to anatomical features within vasculature of a patient (e.g., a known path of a blood vessel within a patient).

Shaped rod 168 can include any suitable material or combination of materials. In some examples, shaped rod 168 includes a different material as compared to other components of endovascular device 160 (e.g., as compared to elongated body 162 of endovascular device 160). In some examples, shaped rod 168 includes a material that is relatively stiffer (e.g., resists bending and/or resists permanent deformation to a greater extent) and/or relatively harder (e.g., has a higher Shore D hardness) as compared to elongated body 162. In some examples, shaped rod 168 defines a Shore D hardness of 55D to 75D (e.g., which may be greater than a Shore D hardness of elongated body 162). In some examples, shaped rod 168 includes a polymer, such as polyurethane. In some examples, shaped rod 168 includes a shape-memory material (e.g., nitinol and/or other suitable shape-memory alloys).

In some examples, shaped rod 168 includes (e.g., is formed from, formed with, and/or is loaded with) a radiopaque or a radiographic material. In examples in which shaped rod 168 includes a radiopaque or a radiographic material, a user (e.g., a clinician) may be able to visualize shaped rod 168 via a suitable medical imaging modality (e.g., x-ray, fluoroscopy, angiography, and/or the like). Visualization of shaped rod 168 via the suitable medical imaging modality can enable a clinician to determine a shape, position, and/or other information about shaped rod 168, as well as coiled portion 130 of endovascular device 160, relative to other structural features of endovascular therapy system 100 and/or anatomical features of a patient (e.g., patient 12 in the example of FIG. 1).

Shaped rod 168 can have any suitable size, shape, form factor, and/or other physical properties. In some examples, one or more of a size, stiffness, form factor, and/or another physical property of shaped rod 168 is selected based on the target anatomy in which endovascular device 160 is positioned. For example, shaped rod 168 can be selected from group of shaped rods having different sizes, stiffnesses, form factors, and/or another physical properties depending on the target anatomy in which endovascular device 160 is positioned. For example, shaped rod 168, including the physical properties thereof, can be selected based on whether the target vasculature is within the brain of a patient (e.g., within the neurovasculature), within a jugular vein, or within another blood vessel. Shaped rod 168 can be shaped prior to and/or after insertion into a lumen of endovascular device 160.

In some examples, shaped rod 168 is configured to receive a guidewire and/or another elongated body therethrough. In some examples, and with reference to FIG. 3C, shaped rod 168 defines a lumen 152. Lumen 152 can be sized, shaped, and/otherwise configured to receive a guidewire and/or another elongated body therethrough.

Coiled portion 130 of endovascular device 160 can include other structural features in addition to and/or instead of shaped rod 168. In some examples, endovascular device 160 includes a jacket having a pre-formed shape (e.g., a pre-formed coil shape), such that the portion of endovascular device 160 including the jacket defines coiled portion 130. The jacket can extend around at least a portion of elongated body 162 of endovascular device 160. In yet other examples, a portion of elongated body 162 of endovascular device 160 can be thermally formed to define coiled portion 130.

In some examples, the endovascular device 160 includes one or more anchors 166 (shown individually as anchor 166A, anchor 166B, and anchor 166C, but collectively referred to herein as anchors 166). As illustrated in the example of FIG. 3A, anchors 166 are positioned on coiled portion 130 of the endovascular device 160. In other examples, one or more of anchors 166 can additionally or alternatively be positioned on another portion of endovascular device 160 (e.g., a portion proximal to and/or distal to coiled portion 130, such as one or more of proximal segment 132 and/or distal segment 134 of endovascular device 160).

In some examples, anchors 166 are configured to anchor endovascular device 160 (e.g., at least elongated body 162 of endovascular device 160) within the vasculature of the patient (e.g., jugular vein 13 of patient 12, as shown in, and described with respect to, FIG. 1). In some examples, and with reference to FIG. 3D and FIG. 3E, each of anchors 166 are configured to contact, be in close proximity to, and/or otherwise engage blood vessel wall 122 of blood vessel 120. Anchors 166 can serve as additional and/or complementary structural features to coiled portion 130 of endovascular device 160. In some examples, anchors 166 are configured to minimize or even prevent forces applied to endovascular device 160 (e.g., axial forces, rotational forces, and/or twisting forces) from being transferred to expandable structure 190 and/or to the electrodes 170.

In some examples, and with reference to the example of FIG. 3E, anchors 166 are configured to hold at least a portion of endovascular device 160 (e.g., coiled portion 130) spaced apart from vessel wall 122 of blood vessel 120. In some examples, the ability of coiled portion 130 to minimize or prevent forces and/or loads from being transferred to expandable structure 190 can become compromised if coiled portion 130 becomes endothelialized prior to expandable structure 190 becoming endothelialized. Thus, in some examples, anchors 166 can slow down and/or prevent at least coiled portion 130 of endovascular device 160 from becoming endothelialized, e.g., by at least holding coiled portion 130 spaced apart from vessel wall 122 of blood vessel 120.

Anchors 166 can include any suitable shape and/or form factor. In some examples, each of anchors 166 define a wing-shape (e.g., a wing branching in one or more directions outward from elongated body 162). In some examples, each of anchors 166 define one or more wing-shaped portions. In some examples, and with reference to FIG. 3C which illustrates a cross-sectional view of endovascular device 160 and anchor 166B, anchors 166 can define a larger cross-sectional dimension and/or extend to a greater radial dimension as compared to elongated body 162 of endovascular device 160. For example, as illustrated in the example of FIG. 3C, elongated body 162 of endovascular device 160 defines a greatest cross-sectional dimension D3 (which may be a diameter in examples in which elongated body 162 defines a circular or substantially circular cross section). In the example of FIG. 3C, anchor 166B defines a greatest cross-sectional dimension D4. In some examples, dimension D4 of anchor 166B is greater than dimension D3 of elongated body 162. In the example of FIG. 3C, elongated body 162 defines a greatest radial dimension R1 (e.g., as measured from a radial center of elongated body 162). In the example of FIG. 3C, anchor 166B defines a greatest radial dimension R2 (e.g., as measured from the radial center of elongated body 162). In some examples, radial dimension R2 of anchor 166B is greater than radial dimension R1 of elongated body 162. As discussed herein, anchor 166B can be representative of each of anchor 166A, anchor 166B, and/or anchor 166C.

In some examples, each of anchors 166 are configured to transform between an anchor delivery configuration (e.g., which may be a relatively low-profile configuration) and an anchor deployed configuration (e.g., which may be an expanded configuration), such as the configuration illustrated in the example of at least FIG. 3C. For example, and with reference to FIG. 3C which illustrates a representative anchor 166B, anchor 166B can be configured to flex to assume a relatively low-profile configuration. In some examples, one or more portions of anchor 166B are configured to flex, bend, and/or otherwise deform toward a more radially inward part of elongated body 162 of endovascular device 160. For example, in some examples, anchor 166B includes one or more wing portions (e.g., the portions of anchor 166B extending in the positive and negative z-axis directions according to the orthogonal x-y-z axes of FIG. 3C), and the one or more wing portions can be configured to flex inward toward a radial center of elongated body 162 (e.g., toward lumen 152 in the example of FIG. 3C). The ability of anchor 166B to assume a relatively low-profile configuration can enable endovascular device 160 to define a relatively low-profile configuration, which may facilitation relatively easier deliverability and navigation of endovascular device 160 through a delivery catheter, delivery sheath, and/or vasculature of a patient.

Anchors 166 can comprise any suitable material or combination of materials. In some examples, anchors 166 include a common material with one or more components of endovascular device 160 (e.g., the outer, elongated body 162 of endovascular device 160). In some examples, anchors 166 include a different material than elongated body 162. In some examples, one or more of anchors 166 include a different polymer material as compared to elongated body 162. In some examples, anchors 166 can be formed from one or more of polyurethane, silicone, and/or a thermoplastic elastomeric polymers (e.g., including thermoplastic elastomeric copolymers). Anchors 166 can include a material that is relatively harder (e.g., has a higher Shore D hardness) than elongated body 162 of elongated body 162. A relatively harder material of anchors 166 as compared to elongated body 162 may enable anchors 166 to better engage vessel wall 122 of blood vessel 120 and/or anchor elongated body 162 of endovascular device 160 within blood vessel 120. In some examples, anchors 166 include a metal (e.g., such as a shape-memory alloy).

In some examples, anchors 166 include one or more of a surface treatment, surface features and/or a surface coating. For example, in some examples, anchors 166 define one or more holes (e.g., through holes and/or blind holes). As another example, anchors 166 can include a surface treatment that increases a surface area of one or more surfaces of one or more of anchors 166. In some examples, the surface treatment can facilitate increased engagement (e.g., contact, friction, and/or the like) between anchors 166 and vessel wall 122 of blood vessel 120. In some examples, the surface coating can include an antithrombogenic coating.

As described herein, elongated body 162 of endovascular device 160 can include a first material, such as a first polymer material. Anchors 166 can include a second material different than the first material of the elongated body 162, such as a second polymer material different than the first polymer material of elongated body 162. The first material of the elongated body 162 can be relatively softer and/or more flexible as compared to the material of anchors 166 such as to enable endovascular device 160 to be navigated through torturous anatomy.

In some examples, anchors 166 include a shape memory material, such as nitinol and/or another suitable shape memory alloy. In examples in which anchors include a metal, anchors 166 can include a wire-form shape (e.g., in which a wire forms the outer perimeter of the shape of anchors shown in the examples of at least FIG. 3A, FIG. 3B, FIG. 3C, FIG. 3D, and/or FIG. 3E).

Anchors 166 can additionally include features that facilitate increased friction and/or engagement with blood vessel wall 122 of blood vessel 120. For example, anchors can include one or more of a surface texturing, a coating, and/or another treatment. The surface texturing, a coating, and/or another treatment can facilitate increased friction and/or engagement between of anchors 166 and blood vessel wall 122.

In some examples, one or more of anchors 166 include a radiopaque or radiographic material. Such radiopaque or radiographic materials can enable anchors 166 to be visible via suitable medical imaging modality (e.g., x-ray, fluoroscopy, angiography, and/or the like). Visualization of anchors 166 via the suitable medical imaging modality can enable a clinician to determine the position of anchors 166 relative to other structural features of endovascular therapy system 100 and/or anatomical features of a patient (e.g., patient 12 in the example of FIG. 1). For example, in some examples, anchors 166 (e.g., when viewed via a suitable medical imaging modality) are configured to indicate a radial expansion of coiled portion 130. In some examples, anchors 166 (e.g., when viewed via a suitable medical imaging modality) are configured to indicate an axial extension (e.g., and/or elongation) of coiled portion 130. In some examples, anchors 166 (e.g., when viewed via a suitable medical imaging modality) are configured to indicate a spacing between coiled portion 130 and expandable structure 190 and/or electrodes 170, which can also be radiopaque or radiographic.

Any suitable number of anchors 166 can be positioned on endovascular device 160 and/or on coiled portion 130 of endovascular device 160 (e.g., one anchor, two anchors, three anchors, four anchors, ten anchors, twenty anchors, or more). In some examples, two or more anchors 166 are positioned on coiled portion 130 of endovascular device 160.

In some examples, anchors 166 are mechanically coupled to and/or integrally formed with a portion of elongated body 162 of endovascular device 160. For example, each of anchors 166 can be a separate component from elongated body 162, and each of anchors 166 can be mechanically coupled to elongated body 162 via suitable a mechanical coupling mechanism. For example, anchors 166 can be mechanically affixed to elongated body 162 via by melted (e.g., reflowed) material of one or more of anchors 166 and/or elongated body 162. In some examples, anchors 166 are injection molded onto elongated body 162. Other additional or alternative mechanical coupling mechanisms for affixing anchors 166 to elongated body 162 include adhesive, crimping, and/or another suitable mechanical coupling mechanism.

In examples in which multiple anchors 166 are positioned on coiled portion 130, each of the multiple anchors 166 can be positioned at respective locations along and/or around central longitudinal coil axis 131 of coiled portion 130. In some examples, two or more of anchors 166 are axially spaced apart along central longitudinal coil axis 131 of coiled portion 130. For example, as illustrated in the example of at least FIG. 3A, each of anchor 166A, anchor 166B, and anchor 166C are positioned at respective axial locations along central longitudinal coil axis 131 of coiled portion 130 (e.g., along the x-axis direction according to the orthogonal x-y-z axes of FIG. 3A). In some examples, two or more of anchors 166 are circumferentially spaced apart around central longitudinal coil axis 131 of coiled portion 130. For example, as illustrated in the example of at least FIG. 3A and FIG. 3D, each of anchor 166A, anchor 166B, and anchor 166C are positioned at respective circumferentially locations around central longitudinal coil axis 131 of coiled portion 130. In such a configuration, each of anchor 166A, anchor 166B, and anchor 166C can face in a unique radial direction outward from central longitudinal coil axis 131. In some examples, as illustrated in FIG. 3D, anchors 166 are equally spaced or substantially equally spaced (e.g., to the extent permitted by manufacturing tolerance) around central longitudinal coil axis 131 of coiled portion 130 (e.g., in a circumferential direction around central longitudinal coil axis 131). For example, each of anchor 166A, anchor 166B, and anchor 166C can be circumferentially spaced apart by about 120 degrees around central longitudinal coil axis 131 of coiled portion 130. In other examples, anchors 166 are unequally spaced apart from adjacent ones of anchors 166 in a circumferential direction around central longitudinal coil axis 131.

In some examples, each of anchors 166 are circumferentially offset from electrodes 170 (e.g., as measured in a circumferential direction around central longitudinal coil axis 131 and/or around central longitudinal axis 191 of expandable structure 190). For example, as illustrated in the example of FIG. 3D, each of anchor 166A, anchor 166B, and anchor 166C are circumferentially spaced apart (e.g., circumferentially offset) from electrodes 170 (e.g., from all of electrodes 170 and/or from the array formed by electrodes 170). In some examples, as illustrated in the example of FIG. 3D, electrodes 170 face in a first radial direction 171 (e.g., which may be a radial direction extending away from central longitudinal axis 191). First radial direction 171 may be a direction that faces target tissue (e.g., one or more nerves, such as vagus nerve 21 of FIG. 1) outside of blood vessel 120. In some examples, as illustrated in the example of FIG. 3D, anchor 166A is positioned on endovascular device 160 such that anchor 166A generally faces in radial direction 167A (e.g., which may be a second radial direction 167A different than first radial direction 171). Anchor 166B is positioned on endovascular device 160 such that anchor 166B generally faces in radial direction 167B (e.g., which may be a third radial direction 167B different than first radial direction 171 and/or second radial direction 167A). Anchor 166C is positioned on endovascular device 160 such that anchor 166C generally faces in radial direction 167C (e.g., which may be a fourth radial direction 167C different than one or more of first radial direction 171, second radial direction 167A, and/or third radial direction 167B). As illustrated in the example of FIG. 3D, one or more of second radial direction 167A, third radial direction 167B, and/or fourth radial direction 167C can be circumferentially spaced apart from first radial direction 171 faced by electrodes 170 (e.g., relative to and/or about central longitudinal axis 191 and/or central longitudinal coil axis 131). As anchors 166 can push on vessel wall 122 and cause vessel wall 122 to slightly expand radially outward (e.g., when elongated body 162 of endovascular device 160 is in the body deployed configuration which can cause anchors 166 to appose and/or push against vessel wall 122), positioning anchors 166 circumferentially offset from electrodes 170 can help ensure electrodes 170 remain in adequate apposition with vessel wall 122. For example, by positioning anchors 166 circumferentially offset from electrodes 170, anchors 166 may have a reduced effect on a portion of vessel wall 122 apposed by electrodes 170.

In some examples, and with reference to FIG. 3D, coiled portion 130 is configured to anchor endovascular device 160 in blood vessel 120. For example, coiled portion 130 can be configured to limit, minimize, and/or even prevent movement (e.g., axial movement and/or rotational movement) of expandable structure 190 and/or electrodes 170 relative to blood vessel 120. In some examples, coiled portion 130 is configured to be in contact and/or remain in contact with blood vessel wall 122 of blood vessel 120. In some examples, coiled portion 130 is configured to remain in relatively close proximity to, but not necessarily always contacting, blood vessel wall 122 of blood vessel 120. In some examples, when a force (e.g., an axial force and/or rotational force) acts directly on expandable structure 190 (e.g., due to blood flow, spasms of the blood vessel wall 122 proximate expandable structure 190, muscles causing deformation of vessel wall 122, and/or other causes), coiled portion 130 can be configured to limit, minimize, and/or even prevent movement (e.g., axial movement and/or rotation) of expandable structure 190 and/or electrodes 170 relative to blood vessel 120. In some examples, coiled portion 130 is configured to provide a counter force to a force applied to expandable structure 190 and/or electrodes 170.

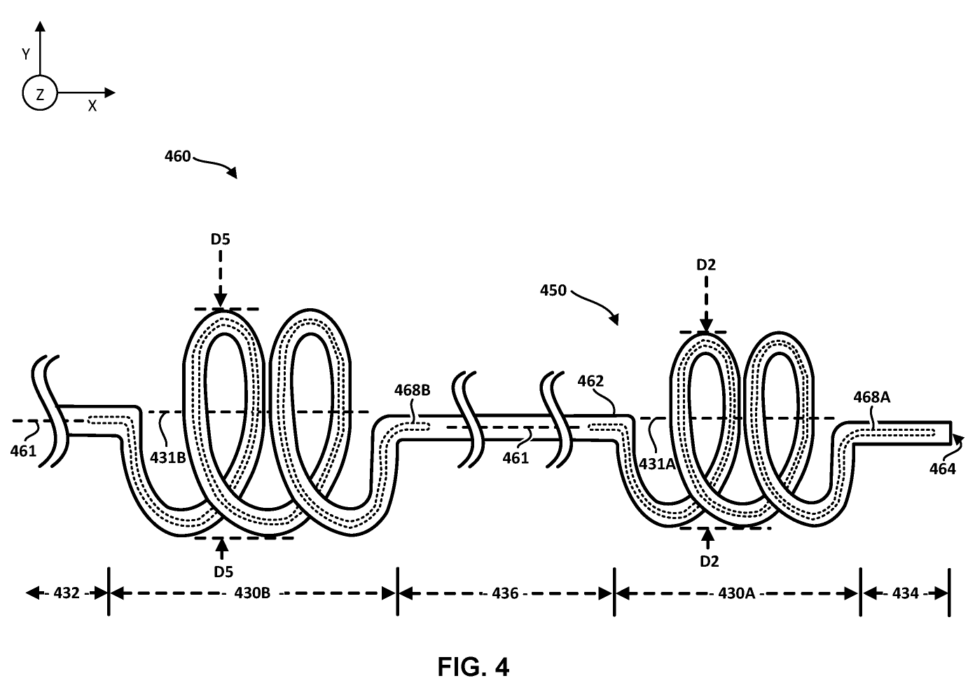

FIG. 4 illustrates an example endovascular device 460, which is an example of endovascular device 16 of FIG. 1 and can be configured similar to endovascular device 160 of FIG. 3A, FIG. 3B, FIG. 3C, FIG. 3D, and/or FIG. 3E except as described herein. Although not shown in the example of FIG. 4, an expandable structure and/or one or more electrodes can be mechanically coupled to a portion of an elongated body 462 of endovascular device 460 (e.g., similar to expandable structure 190 and/or electrodes 170 of FIG. 3A), such as at a distal portion 450 of endovascular device 460.

In some examples, elongated body 462 of endovascular device 460 defines an elongated body central longitudinal axis 461 extending along elongated body 462 of endovascular device 460. Elongated body central longitudinal axis 461 may be a central longitudinal axis of elongated body 462 of endovascular device 460. With reference to FIG. 4, elongated body 462 of endovascular device 460 can extend between an elongated body proximal end (not shown in the examples of FIG. 4) and an elongated body distal end 464.

In some examples, endovascular device 460 includes multiple coiled portions. For example, as illustrated in the example of FIG. 4, elongated body 462 of endovascular device 460 includes at least a first coiled portion 430A and a second coiled portion 430B. Each of first coiled portion 430A and/or second coiled portion 430B can be configured similarly to coiled portion 130 of at least FIG. 3A, except as described herein. For example, each of first coiled portion 430A and/or second coiled portion 430B can be configured to absorb and/or accommodate axial forces (e.g., forces that tend to push or pull on a portion of endovascular device 460, such as elongated body 462), such that the transfer of such forces to an expandable structure and/or one or more electrodes attached to elongated body 462 (e.g., expandable structure 190 and/or electrodes 170 of FIG. 3A) is reduced and/or eliminated. Additionally or alternatively, each of first coiled portion 430A and/or second coiled portion 430B can be configured to accommodate rotational forces and/or twisting forces (e.g., forces that tend cause endovascular device 460 to rotate or twist), such that the transfer of such forces to an expandable structure and/or one or more electrodes attached to endovascular device 460 is reduced and/or eliminated.

While the example of FIG. 4 illustrates elongated body 462 of endovascular device 460 as having two, spaced apart coiled portions, endovascular device 460 can have any suitable number of unique coiled portions (e.g., two coiled portions, three coiled portions, four coiled portions, or more coiled portions).

In some examples, each of first coiled portion 430A and/or second coiled portion 430B can be configured to anchor endovascular device 460 within vasculature (e.g., within a blood vessel, such as blood vessel 120 as illustrated in at least FIG. 3D). For example, as described with respect to coiled portion 130 of at least FIG. 3A and FIG. 3D, each of first coiled portion 430A and/or second coiled portion 430B can be configured to limit, minimize, and/or even prevent movement (e.g., axial movement and/or rotational movement) of an expandable structure and/or electrodes (not illustrated in the example of FIG. 4) attached to elongated body 462.

In some examples, each of first coiled portion 430A and second coiled portion 430B are configured to be positioned in a different portion of the vasculature. In some examples, first coiled portion 430A is configured to be positioned within a first blood vessel, and second coiled portion 430B is configured to be positioned within a second blood vessel different than the first blood vessel. For example, in some examples, first coiled portion 430A is configured to be positioned in a relatively distal portion of the vasculature (e.g., in a first blood vessel, such as a jugular vein 13 of FIG. 1, and/or a carotid artery). In some examples, second coiled portion 430B is configured to be positioned in a more proximal portion of the vasculature as compared to first coiled portion 430A (e.g., in a second blood vessel, such as a subclavian vein or a subclavian artery). In some cases, having both of first coiled portion 430A and second coiled portion 430B can enable relatively better anchoring in the vasculature, e.g., as compared to examples in which endovascular device 460 includes one or no coiled portions. For example, each of first coiled portion 430A and second coiled portion 430B of endovascular device 460 can be sized, shaped, and/or otherwise configured according to the specific blood vessel in which each of first coiled portion 430A and second coiled portion 430B resides, which can help facilitate relatively better anchoring of endovascular device 460 as a whole within the vasculature. While the example of FIG. 4 illustrates endovascular device 460 as having first coiled portion 430A and second coiled portion 430B, endovascular device 460 can include any number of coiled portions configured to be positioned in any number and/or type of blood vessels.

First coiled portion 430A and second coiled portion 430B can have any suitable configuration. First coiled portion 430A and second coiled portion 430B can be spaced apart along endovascular device 160 (e.g., spaced apart along elongated body central longitudinal axis 461 of elongated body 462). In some examples, as illustrated in FIG. 4, first coiled portion 430A and second coiled portion 430B are separated by an intermediate portion 436 of elongated body 462. Intermediate portion 436 can be a straight or substantially straight portion of endovascular device 460 (e.g., of elongated body 462). Intermediate portion 436 can define a sufficient length such that each of first coiled portion 430A and/or second coiled portion 430B reside in a different blood vessel. In some examples, intermediate portion 436 defines a length of greater than 5 cm, such as greater than 10 cm.

As illustrated in at least FIG. 4, first coiled portion 430A (e.g., including a plurality of coil loops that form first coiled portion 430A) can define a central longitudinal coil axis 431A extending through a radial center of first coiled portion 430A. Second coiled portion 430B (e.g., including a plurality of coil loops that form second coiled portion 430B) can define a central longitudinal coil axis 431B extending through a radial center of second coiled portion 430B.

In some examples, elongated body 462 of endovascular device 460 (e.g., distal portion 450 of endovascular device 460) includes a distal segment 434. Distal segment 434 of endovascular device 460 can extend distally of first coiled portion 430A. In some examples, distal segment 434 is configured to mechanically couple to a portion of an expandable structure (e.g., expandable structure 190 of at least FIG. 3A). Distal segment 434 of endovascular device 160 can be straight or substantially straight (e.g., as compared to first coiled portion 430A). Distal segment 434 can be an example of, and configured similarly to, distal segment 134 of at least FIG. 3A and FIG. 3B, except as described herein.

In some examples, elongated body 462 of endovascular device 460 includes a proximal segment 432. Proximal segment 432 of endovascular device 460 can extend proximally of second coiled portion 430B. Proximal segment 432 can be straight or substantially straight (e.g., as compared to coiled portion 130). Proximal segment 432 can extend to a proximal end (e.g., a proximalmost end) of elongated body 462 of endovascular device 460. Proximal segment 432 can be an example of, and configured similarly to, proximal segment 132 of at least FIG. 3A and FIG. 3B, except as described herein.

First coiled portion 430A and second coiled portion 430B can have any suitable shape and/or define any suitable size. In the example of FIG. 4, first coiled portion 430A defines a greatest cross-sectional dimension D2. In some examples, as described with respect to coiled portion 130 of FIG. 3A, dimension D2 is a diameter (e.g., in examples in which first coiled portion 430A defines a circular or substantially circular cross-section). In some examples, as also illustrated in FIG. 4, second coiled portion 430B defines a greatest cross-sectional dimension D5. In some examples, dimension D5 is a diameter (e.g., in examples in which second coiled portion 430B defines a circular or substantially circular cross-section). In some examples, dimension D5 of second coiled portion 430B is different than dimension D2 of first coiled portion 430A. For example, dimension D2 of first coiled portion 430A can correspond to the first blood vessel in which first coiled portion 430A is configured to be positioned and dimension D5 of second coiled portion 430B can correspond to the second blood vessel in which second coiled portion 430B is configured to be positioned. For example, in some examples, dimension D5 of second coiled portion 430B is greater than dimension D2 of first coiled portion 430A (e.g., at least 5 percent greater, such as at least 10 percent greater).

First coiled portion 430A and second coiled portion 430B can define any suitable length. In some examples, first coiled portion 430A and second coiled portion 430B define the same length and/or define a substantially similar length (e.g., as measured in the x-axis direction according to the orthogonal x-y-z axes of FIG. 4). In some examples, first coiled portion 430A and second coiled portion 430B define a different length. For example, in some examples, a length of second coiled portion 430B (e.g., as measured along axis 431B of second coiled portion 430B) is different than (e.g., greater than or less than) a length of first coiled portion 430A (e.g., as measured along axis 431A of first coiled portion 430A). In some examples, a length of first coiled portion 430A is different than (e.g., greater than or less than) a length of second coiled portion 430B by at least 5 percent, such as at least 10 percent). In examples in which first coiled portion 430A and second coiled portion 430B define different lengths, the length of each of first coiled portion 430A and second coiled portion 430B can correspond to the different blood vessel in which each of first coiled portion 430A and second coiled portion 430B are positioned.

Each of first coiled portion 430A and second coiled portion 430B can define unique (e.g., different) coil shapes. For example, first coiled portion 430A can define one or more of a first length, a first number of coil loops, and/or a first coil pitch (e.g., a spacing between adjacent coil loops). Second coiled portion 430B can define one or more of a second length, a second number of coil loops, and/or a second coil pitch (e.g., a spacing between adjacent coil loops). In some examples, the first length, the first number of coil loops, and/or the first coil pitch of first coiled portion 430A can be the same or substantially similar to one or more of the second length, the second number of coil loops, and/or the second coil pitch of second coiled portion 430B, respectively. In other examples, the first length, the first number of coil loops, and/or the first coil pitch of first coiled portion 430A can be different than one or more of the second length, the second number of coil loops, and/or the second coil pitch of second coiled portion 430B, respectively.

In some examples, endovascular device 460 includes one or more structural features that enable each of first coiled portion 430A and second coiled portion 430B to define respective coil shapes. In some examples, as illustrated in the example of FIG. 4, endovascular device 460 (e.g., first coiled portion 430A of endovascular device 460) includes a first shaped rod 468A. First shaped rod 468A can be configured similarly to shaped rod 168 of FIG. 3B, except as described herein. For example, first shaped rod 468A can define a first pre-formed coil shape. First shaped rod 468A can be configured to maintain the coil shape of first coiled portion 430A. First shaped rod 468A, when positioned within and/or along elongated body 462 of endovascular device 460, can be configured to cause elongated body 462 of endovascular device 460 to assume and/or maintain a coil shape such that endovascular device 460 defines first coiled portion 430A.

In some examples, as illustrated in FIG. 4, endovascular device 460 (e.g., second coiled portion 430B of endovascular device 460) includes a second shaped rod 468B. Second shaped rod 468B can define a second pre-formed coil shape (e.g., different than the first pre-formed coil shape corresponding to first shaped rod 468A). Second shaped rod 468B can be configured to maintain the coil shape of second coiled portion 430B. Second shaped rod 468B, when positioned within and/or along elongated body 462 of endovascular device 460, can be configured to cause elongated body 462 of endovascular device 460 to assume and/or maintain a coil shape such that endovascular device 460 defines second coiled portion 430B.

In some examples, second shaped rod 468B is physically separate of and spaced apart from first shaped rod 468A (e.g., when each of second shaped rod 468B and first shaped rod 468A are positioned within elongated body 462 of endovascular device 460). In other examples, second shaped rod 468B and first shaped rod 468A are formed from a continuous shaped rod having spaced apart pre-formed coil shapes (e.g., corresponding to each of first coiled portion 430A and second coiled portion 430B).

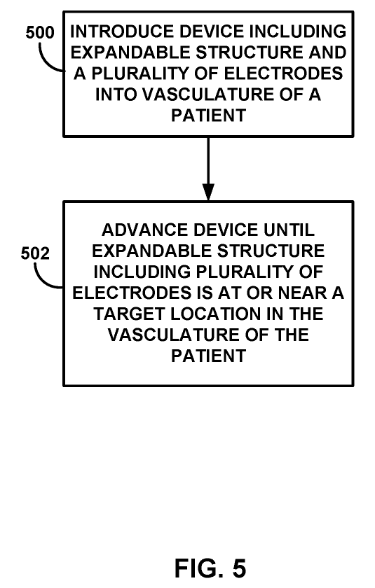

FIG. 5 is a flow diagram illustrating an example technique for using an endovascular device and systems according to the techniques of this disclosure, which may include placing an endovascular device (e.g., which may be and/or include a medical lead) adjacent a target location in vasculature of a patient. The technique of FIG. 5 is described with respect to therapy system 10 of FIG. 1, as well as endovascular therapy system 100 of at least FIG. 3A (which is an example of therapy system 10 of FIG. 1), but may be used with any of the device, systems, and/or elements of systems described in this disclosure.

In the example of FIG. 5, the technique includes introducing an endovascular device (e.g., endovascular device 16 and/or endovascular device 160) into vasculature of patient 12 (500). For example, a user (e.g., a clinician) may introduce at least distal portion 15 of endovascular device 16 through an access point in patient 12 including a femoral artery access point or radial artery access point. In some examples, one or more of an introducer sheath, a guide catheter, and/or a guidewire is used to facilitate introduction of endovascular device 16 into patient 12.

In the example of FIG. 5, the technique further includes advancing endovascular device 16 through the vasculature of patient 12 until electrodes 17 are adjacent a target location in the vasculature of patient 12 (502). In some examples, a clinician advances endovascular device 16 through vasculature of patient 12 until electrodes 17 are located within jugular vein 13 and positioned adjacent vagus nerve 21. In other examples, a clinician advances endovascular device 16 through vasculature of patient 12 until electrodes 17 are located within a cranial blood vessel proximate one or more target brain structures.

In some examples, the technique further includes causing endovascular device 16 and/or endovascular device 160 including coiled portion 130 of elongated body 162 to transform between the body delivery configuration and the body deployed configuration (e.g., which may be an expanded configuration of endovascular device 160 in which endovascular device 160 defines coiled portion 130). Causing elongated body 162 including coiled portion 130 to transform from the body delivery configuration to the body deployed configuration can include removing a straightening element from endovascular device 160 (e.g., such as a wire that causes coiled portion 130 to assume an uncoiled shape) and/or advancing coiled portion 130 distally of a sheath or other elongated body surrounding at least coiled portion 130. In some examples, as discussed with respect to at least FIG. 3A and FIG. 3D, endovascular device 160 includes at least one coiled portion 130. Coiled portion 130 can be configured to limit or prevent a force (e.g., an axial force, rotational force, twisting force, and/or the like) applied to elongated body 162 of endovascular device 160 from being transferred to expandable structure 190 and/or the electrodes 170. In some examples, as illustrated in at least FIG. 3D, coiled portion 130 is configuration to anchor endovascular device 160 in blood vessel 120 (e.g., before, during, and/or after deployment of expandable structure 190 to the deployed configuration).

In examples in which endovascular device 160 includes two or more coiled portions (e.g., endovascular device 460 as discussed with respect to FIG. 4, which includes first coiled portion 430A and second coiled portion 430B), the technique can include positioning a first coiled portion (e.g., first coiled portion 430A) within a first blood vessel, and positioning the second coiled portion (e.g., second coiled portion 430B) within a second blood vessel different than the first blood vessel. As discussed with respect to the example of FIG. 4, having two or more coiled portions (e.g., that are each differently configured for different blood vessels) can enable relatively better anchoring of endovascular device 460 as a whole in the vasculature, e.g., as compared to examples in which endovascular device 460 includes one or no coiled portions.

In some examples, expandable structure 190, which can be at a distal portion of elongated body 162 of endovascular device 160, is configured transform from a relatively low-profile delivery configuration to a deployed configuration in a blood vessel of a patient (e.g., within jugular vein 13 of patient 12). In some examples, expandable structure 190 remains in the delivery configuration during advancement of endovascular device 160 through the vasculature.

In some examples, the technique includes causing the expandable structure 190 to transform to the deployed (e.g., expanded) configuration once electrodes 170 are adjacent the target site. In the deployed configuration of expandable structure 190, one or more of electrodes 170 can be positioned into apposition with the vessel wall (e.g., the vessel wall of jugular vein 13). In some examples, electrodes 170 are configured to bias transmissions of electrical signals to tissue surrounding the blood vessel (e.g., jugular vein 13) as compared to radially inward from the blood vessel wall (e.g., towards central longitudinal axis 191). In some examples, electrodes 170 are configured to bias sensing of electrical signals from tissue surrounding the blood vessel (e.g., jugular vein 13) as compared to radially inward from the blood vessel wall (e.g., towards central longitudinal axis 191). The biasing of transmissions of electrical signals to and/or from tissue surrounding the blood vessel can help facilitate directional electrical stimulation and/or sensing. In this way, the endovascular devices described herein can help target any suitable target tissue sites (e.g., nerve structures and/or brain structures) from an endovascular location and/or help avoid nontarget tissue sites, e.g., those associated with negative side effects.

After electrodes 170 are adjacent the target location (e.g., vagus nerve 21, other nerve, or one or more brain structures), the method can include initiating (e.g., via programmer 20, or another suitable device) electrical stimulation therapy and/or sensing of one or more patient parameters by medical device 14 via electrodes 170.

In some examples, one or more elements of therapy system 100 are configured to facilitate positioning of electrodes 170 at the target site (e.g., via radiographic and/or radiopaque portions that indicate a positioning of electrodes 170). In some examples, at least a portion of therapy system 100 is aligned with one or more of electrodes 170 (and/or circumferentially aligned an array of electrodes formed from a group of electrodes 170) to indicate a direction (e.g., a radial direction) faced by electrodes 170. In some examples, endovascular therapy system 100 (e.g., one or more components of endovascular therapy system 100) includes a radiographic or radiopaque marker that is circumferentially aligned with one or more of electrodes 170 and/or an electrode array formed by electrodes 170. In some examples, one or more portions of endovascular device 160 (e.g., elongated body 162 and/or expandable structure 190) includes a radiographic or radiopaque material circumferentially aligned with electrodes 170 and configured to indicate a radial direction (e.g., a radial direction outwards from central longitudinal axis 191) faced by electrodes 170 (e.g., when expandable structure 190 is in the deployed configuration).

This disclosure includes the following non-limiting examples.

Example 1: An endovascular device includes an elongated body configured to be introduced into vasculature of a patient; an expandable structure at a distal portion of the elongated body; and a plurality of electrodes carried by the expandable structure, wherein the elongated body is configured to transform between a body delivery configuration and a body deployed configuration, and wherein in the body deployed configuration, the distal portion of the elongated body defines a coiled portion, the coiled portion located proximal of the expandable structure and configured to limit or prevent an axial force applied to the elongated body from being transferred to the expandable structure or the plurality of electrodes.

Example 2: The endovascular device of example 1, further comprising one or more anchors positioned on the elongated body, each anchor of the one or more anchors configured to engage a vessel wall of the vasculature when the elongated body is positioned within the vasculature of the patient.

Example 3: The endovascular device of example 2, wherein the one or more anchors are positioned on the coiled portion of the elongated body.

Example 4: The endovascular device of any of examples 2 and 3, wherein the elongated body includes a first material, and wherein the one or more anchors include a second material different from the first material.

Example 5: The endovascular device of example 4, wherein the second material is a polymer.

Example 6: The endovascular device of example 4, wherein the second material is radiopaque or radiographic.

Example 7: The endovascular device of any of examples 2 through 6, wherein the one or more anchors include two or more anchors positioned on the coiled portion of the elongated body.

Example 8: The endovascular device of example 7, wherein the coiled portion of the elongated body defines a central longitudinal coil axis extending through a radial center of the coiled portion, and wherein the two or more anchors are axially spaced apart along the central longitudinal coil axis in the body deployed configuration.

Example 9: The endovascular device of example 7, wherein the coiled portion of the elongated body defines a central longitudinal coil axis extending through a radial center of the coiled portion, and wherein the two or more anchors are circumferentially spaced apart around the central longitudinal coil axis in the body deployed configuration.

Example 10: The endovascular device of any of examples 1 through 9, wherein the coiled portion includes at least two coil loops.

Example 11: The endovascular device of any of examples 1 through 10, wherein the coiled portion includes a shaped rod positioned within the elongated body, the shaped rod configured to maintain a coil shape of the coiled portion.

Example 12: The endovascular device of example 11, wherein the shaped rod is loaded with a radiopaque or a radiographic material.

Example 13: The endovascular device of any of examples 1 through 12, wherein the coiled portion is a first coiled portion, and wherein the elongated body defines a second coiled portion, the second coiled portion spaced apart from the first coiled portion.

Example 14: The endovascular device of example 13, wherein the first coiled portion is configured to be positioned within a first blood vessel, and wherein the second coiled portion is configured to be positioned within a second blood vessel different than the first blood vessel.

Example 15: The endovascular device of example 14, wherein the first blood vessel is a jugular vein or a carotid artery, and wherein the second blood vessel is a subclavian vein or a subclavian artery.

Example 16: The endovascular device of any of examples 1 through 15, wherein in the body delivery configuration, the elongated body defines a relatively lower profile as compared to the body deployed configuration.

Example 17: The endovascular device of any of examples 1 through 16, wherein in the body delivery configuration, the elongated body does not define the coiled portion.

Example 18: The endovascular device of any of examples 1 through 17, wherein the expandable structure includes a plurality of interconnected struts and is configured to transform from a relatively low-profile delivery configuration to a deployed configuration, and wherein in the deployed configuration, the expandable structure including the plurality of interconnected struts is expanded radially outward as compared to the relatively low-profile delivery configuration to position the plurality of electrodes to deliver electrical stimulation to tissue of the patient or sense a patient parameter from a location within the vasculature of the patient.

Example 19: A method includes introducing an endovascular device into vasculature of a patient, the endovascular device includes an elongated body configured to be introduced into the vasculature of the patient; an expandable structure at a distal portion of the elongated body; and a plurality of electrodes carried by the expandable structure, wherein the elongated body is configured to transform between a body delivery configuration and a body deployed configuration, and wherein in the body deployed configuration, the distal portion of the elongated body defines a coiled portion, the coiled portion located proximal of the expandable structure and configured to limit or prevent an axial force applied to the elongated body from being transferred to the expandable structure or the plurality of electrodes; and advancing the endovascular device until the plurality of electrodes are at or near a target location in the vasculature of the patient.

Example 20: The method of example 19, wherein the endovascular device further comprises one or more anchors positioned on the elongated body, each anchor of the one or more anchors configured to engage a vessel wall of the vasculature when the elongated body is positioned within the vasculature of the patient.

Example21: The method of example 20, wherein the one or more anchors are positioned on the coiled portion of the elongated body.

Example 22: The method of any of examples 20 and 21, wherein the elongated body includes a first material, and wherein the one or more anchors include a second material different from the first material.

Example 23: The method of example 22, wherein the second material is a polymer.

Example 24: The method of example 22, wherein the second material is radiopaque or radiographic.

Example 25: The method of any of examples 20through 24, wherein the one or more anchors include two or more anchors positioned on the coiled portion of the elongated body.

Example 26: The method of example 25, wherein the coiled portion of the elongated body defines a central longitudinal coil axis extending through a radial center of the coiled portion, and wherein the two or more anchors are axially spaced apart along the central longitudinal coil axis in the body deployed configuration.

Example 27: The method of example 25, wherein the coiled portion of the elongated body defines a central longitudinal coil axis extending through a radial center of the coiled portion, and wherein the two or more anchors are circumferentially spaced apart around the central longitudinal coil axis in the body deployed configuration.

Example 28: The method of any of examples 19 through 27, wherein the coiled portion includes at least two coil loops.

Example 29: The method of any of examples 19 through 28, wherein the coiled portion includes a shaped rod positioned within the elongated body, the shaped rod configured to maintain a coil shape of the coiled portion.

Example 30: The method of example 29, wherein the shaped rod is loaded with a radiopaque or a radiographic material.

Example 31: The method of any of examples 19 through 30, wherein the coiled portion is a first coiled portion, and wherein the elongated body defines a second coiled portion, the second coiled portion spaced apart from the first coiled portion.

Example 32: The method of example 31, further includes positioning the first coiled portion within a first blood vessel, and positioning the second coiled portion within a second blood vessel different than the first blood vessel.

Example 33: The method of example 32, wherein the first blood vessel is a jugular vein or a carotid artery, and wherein the second blood vessel is a subclavian vein or a subclavian artery.

Example 34: The method of any of examples 19 through 33, wherein in the body delivery configuration, the elongated body defines a relatively lower profile as compared to the body deployed configuration.

Example 35: The method of any of examples 19 through 34, wherein in the body delivery configuration, the elongated body does not define the coiled portion.

Example 36: The method of any of examples 19 through 35, wherein the expandable structure includes a plurality of interconnected struts and is configured to transform from a relatively low-profile delivery configuration to a deployed configuration, and wherein in the deployed configuration, the expandable structure including the plurality of interconnected struts is expanded radially outward as compared to the relatively low-profile delivery configuration to position the plurality of electrodes to deliver electrical stimulation to tissue of the patient or sense a patient parameter from a location within the vasculature of the patient.

Example 37: An endovascular device includes an elongated body configured to be introduced into vasculature of a patient; an expandable structure at a distal portion of the elongated body; a plurality of electrodes carried by the expandable structure; and two or more anchors positioned on a coiled portion of the elongated body, each anchor of the two or more anchors configured to engage a vessel wall of the vasculature when the elongated body is positioned within the vasculature of the patient, wherein the elongated body is configured to transform between a body delivery configuration and a body deployed configuration, and wherein in the body deployed configuration, the distal portion of the elongated body defines the coiled portion, the coiled portion located proximal of the expandable structure and configured to limit or prevent an axial force applied to the elongated body from being transferred to the expandable structure or the plurality of electrodes.

Example 38: The endovascular device of example 37, wherein the coiled portion of the elongated body defines a central longitudinal coil axis extending through a radial center of the coiled portion, and wherein the two or more anchors are axially spaced apart along the central longitudinal coil axis in the body deployed configuration.

Example 39: The endovascular device of example 37, wherein the coiled portion of the elongated body defines a central longitudinal coil axis extending through a radial center of the coiled portion, and wherein the two or more anchors are circumferentially spaced apart around the central longitudinal coil axis in the body deployed configuration.

The techniques described in this disclosure, including those attributed to medical device 14, programmer 20, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as clinician or patient programmers, medical devices, or other devices. Processing circuitry, control circuitry, and sensing circuitry, as well as other processors and controllers described herein, may be implemented at least in part as, or include, one or more executable applications, application modules, libraries, classes, methods, objects, routines, subroutines, firmware, and/or embedded code, for example. In addition, analog circuits, components and circuit elements may be employed to construct one, some or all of the processing circuitry 30, instead of or in addition to the partially or wholly digital hardware and/or software described herein. Accordingly, analog or digital hardware may be employed, or a combination of the two.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. The computer-readable medium may be an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium stores data that can, over time, change (e.g., in RAM or cache).

The functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements.

Various examples of the disclosure have been described. Any combination of the described systems, operations, or functions is contemplated. These and other examples are within the scope of the following claims.

What is claimed is:

1. An endovascular device comprising:
an elongated body configured to be introduced into vasculature of a patient;
an expandable structure at a distal portion of the elongated body; and
a plurality of electrodes carried by the expandable structure,
wherein the elongated body is configured to transform between a body delivery configuration and a body deployed configuration, and
wherein in the body deployed configuration, the distal portion of the elongated body defines a coiled portion, the coiled portion located proximal of the expandable structure and configured to limit or prevent an axial force applied to the elongated body from being transferred to the expandable structure or the plurality of electrodes.

2. The endovascular device of claim 1, further comprising one or more anchors positioned on the elongated body, each anchor of the one or more anchors configured to engage a vessel wall of the vasculature when the elongated body is positioned within the vasculature of the patient.

3. The endovascular device of claim 2, wherein the one or more anchors are positioned on the coiled portion of the elongated body.

4. The endovascular device claim 2,
wherein the elongated body includes a first material, and
wherein the one or more anchors include a second material different from the first material.

5. The endovascular device of claim 4, wherein the second material is a polymer.

6. The endovascular device of claim 4, wherein the second material is radiopaque or radiographic.

7. The endovascular device of claim 2, wherein the one or more anchors include two or more anchors positioned on the coiled portion of the elongated body.

8. The endovascular device of claim 7,
wherein the coiled portion of the elongated body defines a central longitudinal coil axis extending through a radial center of the coiled portion, and
wherein the two or more anchors are axially spaced apart along the central longitudinal coil axis in the body deployed configuration.

9. The endovascular device of claim 7,
wherein the coiled portion of the elongated body defines a central longitudinal coil axis extending through a radial center of the coiled portion, and
wherein the two or more anchors are circumferentially spaced apart around the central longitudinal coil axis in the body deployed configuration.

10. The endovascular device of claim 1, wherein the coiled portion includes at least two coil loops.

11. The endovascular device of claim 1, wherein the coiled portion includes a shaped rod positioned within the elongated body, the shaped rod configured to maintain a coil shape of the coiled portion.

12. The endovascular device of claim 11, wherein the shaped rod is loaded with a radiopaque or a radiographic material.

13. The endovascular device of claim 1,
wherein the coiled portion is a first coiled portion, and
wherein the elongated body defines a second coiled portion, the second coiled portion spaced apart from the first coiled portion.

14. The endovascular device of claim 13,
wherein the first coiled portion is configured to be positioned within a first blood vessel, and
wherein the second coiled portion is configured to be positioned within a second blood vessel different than the first blood vessel.

15. The endovascular device of claim 14,
wherein the first blood vessel is a jugular vein or a carotid artery, and
wherein the second blood vessel is a subclavian vein or a subclavian artery.

16. The endovascular device of claim 1, wherein in the body delivery configuration, the elongated body defines a relatively lower profile as compared to the body deployed configuration.

17. The endovascular device of claim 1, wherein in the body delivery configuration, the elongated body does not define the coiled portion.

18. The endovascular device of claim 1,
wherein the expandable structure includes a plurality of interconnected struts and is configured to transform from a relatively low-profile delivery configuration to a deployed configuration, and
wherein in the deployed configuration, the expandable structure including the plurality of interconnected struts is expanded radially outward as compared to the relatively low-profile delivery configuration to position the plurality of electrodes to deliver electrical stimulation to tissue of the patient or sense a patient parameter from a location within the vasculature of the patient.

19. An endovascular device comprising:
an elongated body configured to be introduced into vasculature of a patient;
an expandable structure at a distal portion of the elongated body;
a plurality of electrodes carried by the expandable structure; and
two or more anchors positioned on a coiled portion of the elongated body, each anchor of the two or more anchors configured to engage a vessel wall of the vasculature when the elongated body is positioned within the vasculature of the patient,
wherein the elongated body is configured to transform between a body delivery configuration and a body deployed configuration, and
wherein in the body deployed configuration, the distal portion of the elongated body defines the coiled portion, the coiled portion located proximal of the expandable structure and configured to limit or prevent an axial force applied to the elongated body from being transferred to the expandable structure or the plurality of electrodes.

20. The endovascular device of claim 19,
wherein the coiled portion of the elongated body defines a central longitudinal coil axis extending through a radial center of the coiled portion, and
wherein the two or more anchors are axially spaced apart along the central longitudinal coil axis in the body deployed configuration.
